(19) United States
(12) Patent Application Publication
(10) Pub. No.: US 2006/0058247 A1
(43) Pub. Date: Mar. 16, 2006

(54) Methods and compositions for treating cystic fibrosis

(76) Inventors: Yujiro Hata, Cambridge, MA (US); Yat Sun Or, Watertown, MA (US)

Correspondence Address: ELMORE PATENT LAW GROUP, PC, 209 MAIN STREET, N. CHELMSFORD, MA, 01863, US

(21) Appl. No.: 11/226,073

(22) Filed: Sep. 14, 2005

Related U.S. Application Data
(60) Provisional application No. 60/610,427, filed on Sep. 16, 2004.

(51) Int. Cl. A61K 31/7048; A61K 31/496; A61K 31/4709; A61K 31/56

(52) U.S. Cl. 514/28; 514/29; 514/171; 514/253.08; 514/312; 514/154

(57) ABSTRACT

The present invention provides methods of using improved macrolide compositions in the treatment of cystic fibrosis. The macrolide compounds of Formulas 1-21 described herein may be used alone or in a cotherapeutic regimen with other agents that are active against cystic fibrosis. The present invention also provides compositions and methods for treating inflammatory conditions.

### RELATED APPLICATION

This application claims the benefit of U.S. Provisional Application No. 60/610,427, filed on Sep. 16, 2004. The entire teachings of the above application are incorporated herein by reference.

### BACKGROUND OF THE INVENTION

Cystic fibrosis (CF) is a disease that is characterized by chronic infection and inflammation of the respiratory system. It is unclear whether inflammation precedes infection or is caused by infection, but in any case, the inflammation is chronic. Chronic inflammation results in damage to the epithelial cell layer, which then permits Pseudomonas species to colonize the respiratory tract. The cycle of infection and inflammation combined with the other factors associated with CF such impaired mucociliary clearance, and increased sputum viscosity lead to increasing morbidity in the CF patient.

The life expectancy of CF patients has greatly improved due to the aggressive prophylactic and therapeutic use of antibiotics. Attention has turned more recently to the possible anti-inflammatory effects of macrolide antibiotics in treating CF.

Macrolides are a group of antibiotics derived from Streptomyces species. They have a common macrolytic lactone ring to which one or more sugars are attached. Erythromycin is the most widely used macrolide. Azithromycin, and clarithromycin are semi-synthetic macrolides similar in structure to erythromycin.

Macrolides have been used successfully to treat Diffuse Pan-Bronchitis (DPB), a condition that is common in Japan and shares some similarities with CF. A number of human clinical studies have been conducted showing that the use of macrolides such as azithromycin provide a beneficial effect in a proportion of patients with cystic fibrosis.

The mechanisms of action that macrolides use to modulate diseases such as DPB and CF are not fully known, however it is believed that there may be more than one mechanism by which the macrolides function to successfully modulate a disease such as CF. Several studies have indicated that macrolides possess important anti-inflammatory activity which appears to be mediated by an inhibition of neutrophil chemotaxis, reductin of neturophil elastase, and modification of pro-inflammatory cytokines with suppression of interleukin (IL)-1β, IL-6, IL-8 and tumor necrosis factor (TNF) production. Other studies have shown that macrolides may reduce sputum viscoelasticity and airway adhesion of P. aeruginosa and increase killing of mucoid P. aeruginosa, a mechanism that may be mediated by the macrolide's ability to disrupt the integrity of the protective biofilm and impair the transformation of non-mucoid P. aeroginosa to the more virulent mucoid phenotype. In yet other studies, there are indications that macrolides may improve ion transport by upregulating a protein of the same superfamily as cystic fibrosis transmembrane regulater (CFTR), the multidrug resistant protein (MDR). There are also indications that macrolides facilitate broncodialation, perhaps by inhibition of cholinergic responses in the human airway smooth muscle.

In view of the growing evidence in support of the use of macrolides in the treatment of CF, compositions and methods employing improved macrolides with greater efficacy in the treatment of CF and minimal side effects, are desirable.

### SUMMARY OF THE INVENTION

The present invention provides methods of using improved macrolide compositions in the treatment of cystic fibrosis. In one embodiment, the invention provides methods for treating cystic fibrosis in a patient in need thereof comprising administering a therapeutically effective amount of a pharmaceutical composition comprising at least one compound of a Formulas 1-21 described below.

The invention further provides co-therapeutic regimens for treating CF comprising administering simultaneously or sequentially over a period of minutes, hours or days, a therapeutically effective amount of a pharmaceutical composition comprising at least one compound of Formulas 1-21, in combination with therapeutically effective amounts of at least one other therapeutic agent that is effective in treating CF.

The invention also provides methods for treating inflammation in a patient in need thereof comprising administering to a patient a therapeutically effective amount of a pharmaceutical composition comprising at least one compound of Formulas 1-21. In one embodiment, the method of the invention comprises a co-therapeutic treatment regimen for treating inflammation comprising administering, either simultaneously or sequentially, over a period of minutes, hours or days, a therapeutically effective amount of a composition comprising at least one compound of Formulas 1-21 in combination with therapeutically effective amounts of other agents that are active against inflammation.

In one embodiment, the present invention provides methods for treating cystic fibrosis using compounds represented by Formulas 1 and 2, or a pharmaceutically acceptable salt, ester or prodrug thereof.

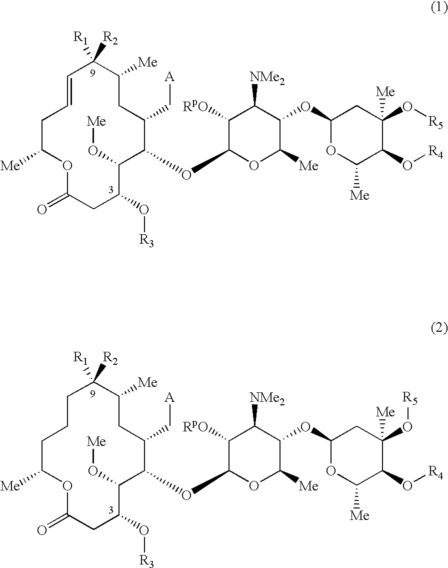

In Formulas 1 and 2:
A is:
(1) CHO;
(2) CH₂—X, where X is selected from the group consisting of
   a. hydroxy or protected hydroxy,
   b. halogen,
   c. NR7R8 wherein R7 and R8 are each independently selected from hydrogen and C1-C6-alkyl, optionally substituted with aryl or heterocyclic groups; or R7 and R8 taken together with the nitrogen atom to which they are connected form a 3- to 7-membered ring which, may optionally contain a hetero function selected from the group consisting of —O—, —NH—, —N(C1-C6-alkyl)-, —N(aryl)-, —N(heteroaryl)-, —S—, —S(O)— and —S(O)2—,
   d. NR7C(O)—R9, where R7 is as previously defined and R9 is selected from the group consisting of
      i. C1-C6-alkyl, optionally substituted with aryl, substituted aryl, heterocyclic or substituted heterocyclic,
      ii. aryl,
      iii. substituted aryl,
      iv. heterocyclic, and
      v. substituted heterocyclic
   e. S(O)ₙ—(C1-C6-alkyl), optionally substituted with aryl or a heterocyclic group where n=0, 1 or 2,
   f. S(O)ₙ-(aryl or heterocyclic group) where n=0, 1 or 2, and
   g. O-(aryl or heterocyclic group);
(3) an aldehyde protecting group,
(4) substituted or unsubstituted imidazole, arylimidazole or heteroarylimidazole,
(5) substituted or unsubstituted oxazole, aryloxazole or heteroaryloxazole,
(6) substituted or unsubstituted thioxazole, arylthioxazole or heteroarylthioxazole,
(7) substituted or unsubstituted imidazoline, arylimidazoline or heteroarylimidazoline,
(8) substituted or unsubstituted oxazoline, aryloxazoline or heteroaryloxazoline, or
(9) substituted or unsubstituted thioxazoline, arylthioxazoline or heteroarylthioxazoline.
   R1 and R2 are each independently selected from the group consisting of
(1) hydrogen,
(2) hydroxy,
(3) protected hydroxy,
(4) OC(O)—C1-C12 alkyl, optionally substituted with aryl, substituted aryl, heterocyclic, substituted heterocyclic,
(5) O—R7 where R7 is as previously defined,
(6) halogen, and
(7) R1 and R2 taken together are oxo;
   R3, R4 and R5 are each independently selected from the group consisting of
(1) hydrogen,
(2) a hydroxy protecting group, and
(3) C(O)—C1-C12-alkyl, optionally substituted with aryl, substituted aryl, heterocyclic, substituted heterocyclic, O—R7 or NR7R8 where R7 and NR7R8 are as previously defined; and
   R^{p} is hydrogen or a hydroxy protecting group.
   A detailed description of the compounds of Formulas 1 and 2 and their synthesis is found in U.S. Pat. No. 6,440,942, incorporated herein by reference.

In another embodiment, the present invention provides methods of treating cystic fibrosis using the compounds of Formulas 3 and 4, or a pharmaceutically acceptable salt, ester or prodrug thereof:

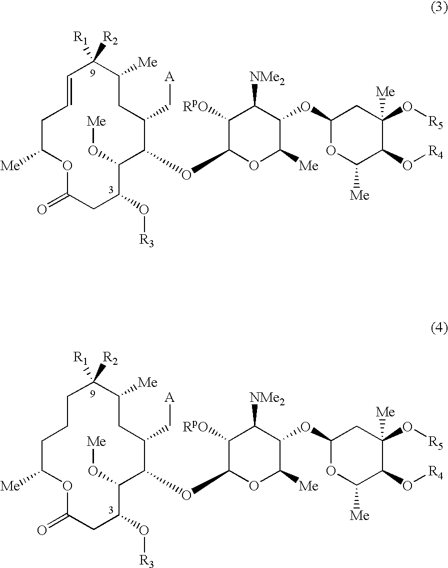

In Formulas 3 and 4:
A is selected from the group consisting of;
(1) —CHO or a protected aldehyde
(2) —CH₂X, where X is selected from the group consisting of;
   a. hydroxy or protected hydroxy,
   b. halogen,
   c. —NR7R8, wherein R7 and R8 are each independently selected from hydrogen, aryl, substituted aryl, heterocyclic, substituted heterocyclic, C1-C6-alkyl, optionally substituted with aryl, substituted aryl, heterocyclic or substituted heterocyclic, C2-C6-alkenyl, optionally substituted with aryl, substituted aryl, heterocyclic or substituted heterocyclic and C2-C6-alkynyl, optionally substituted with aryl, substituted aryl, heterocyclic or substituted heterocyclic; or R7 and R8 taken together with the nitrogen atom to which they are connected form a 3- to 7-membered ring which, may optionally contain a hetero function selected from the group consisting of —O—, —NH—, —N(C1-C6-alkyl)-, —N(aryl)-, —N(heteroaryl)-, —S—, —S(O)— and —S(O)2—,
   d. NR7C(O)—R9, where R7 is as previously defined and R9 is selected from the group consisting of;
      i. C1-C6-alkyl, optionally substituted with aryl, substituted aryl, heterocyclic or substituted heterocyclic,
      ii. aryl,
      iii. substituted aryl,
      iv. heterocyclic, and
      v. substituted heterocyclic
   e. —S(O)ₙ—(C1-C6-alkyl), optionally substituted with aryl substituted aryl, heterocyclic, or substituted heterocyclic, where n=0, 1 or 2,
   f. —S(O)ₙ—(C2-C6-alkenyl), optionally substituted with aryl, substituted aryl, heterocyclic or substituted heterocyclic, where n is as previously defined,
   g. —S(O)ₙ—(C2-C6-alkynyl), optionally substituted with aryl, substituted aryl, heterocyclic or substituted heterocyclic, where n is as previously defined,
   h. —S(O)ₙ-(aryl or heterocyclic), where n is as previously defined,
   i. —O-(aryl or heterocyclic),
(3) substituted or unsubstituted imidazole, arylimidazole or heteroarylimidazole,
(4) substituted or unsubstituted oxazole, aryloxazole or heteroaryloxazole,
(5) substituted or unsubstituted thioxazole, arylthioxazole or heteroarylthioxazole,
(6) substituted or unsubstituted imidazoline, arylimidazoline or heteroarylimidazoline,
(7) substituted or unsubstituted oxazoline, aryloxazoline or heteroaryloxazoline, and
(8) substituted or unsubstituted thioxazoline, arylthioxazoline or heteroarylthioxazoline,
   One of R1 and R2 is hydrogen and the other is —NR7R8 where R7 and R8 are as previously defined;
   R3, R4 and R5 are each independently selected from the group consisting of;
(1) hydrogen,
(2) a hydroxy protecting group, and
(3) —C(O)—(C1-C12-alkyl), optionally substituted with aryl, substituted aryl, heterocyclic, substituted heterocyclic, —O—R7 or —NR7R8 where R7 and R8 are as previously defined; and
   R^{p} is hydrogen or a hydroxy protecting group.
   A detailed description of the compounds of Formulas 3 and 4 and their synthesis is found in U.S. Pat. No. 6,436,906, incorporated herein by reference.

In another embodiment, the present invention provides methods of treating cystic fibrosis using the compounds of Formulas 5 or 6, shown below or a pharmaceutically acceptable salt, ester or prodrug thereof. 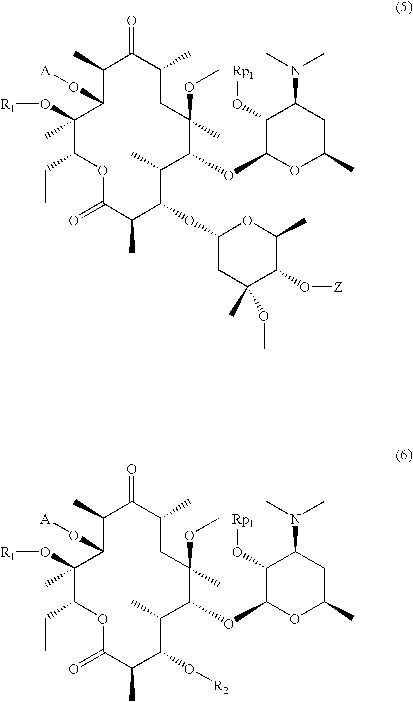
In formulae (5) and (6) above:
A is selected from the group consisting of:
   (1) C₁-C₆-alkyl, C₂-C₆-alkenyl or C₂-C₆-alkynyl, optionally substituted with one or more substituents selected from the group consisting of:
      i. halogen;
      ii. aryl;
      iii. substituted aryl;
      iv. heteroaryl;
      v. substituted heteroaryl;
      vi. —O—R₅, where R₅ is selected from the group consisting of:
         a. hydrogen;
         b. aryl;
         c. substituted aryl;
         d. heteroaryl; and
         e. substituted heteroaryl; and
      vii. —O—C₁-C₆-alkyl-R₅, where R₅ is as previously defined;
      viii. —O—C₂-C₆-alkenyl-R₅, where R₅ is as previously defined;
      ix. —O—C₂-C₆-alkynyl-R₅, where R₅ is as previously defined; and
      x. —NR₆R₇, where R₆ and R₇ are each independently selected from hydrogen, C₁-C₆-alkyl, optionally substituted with one or more substituents selected from the group consisting of halogen, aryl, substituted aryl, heterocyclic and substituted heterocyclic, C₂-C₆-alkenyl, optionally substituted with one or more substituents selected from the group consisting of halogen, aryl, substituted aryl, heterocyclic and substituted heterocyclic, C₂-C₆-alkynyl, optionally substituted with one or more substituents selected from the group consisting of halogen, aryl, substituted aryl, heterocyclic and substituted heterocyclic or R₆R₇ taken with the nitrogen atom to which they are connected form a 3- to 7-membered ring which may optionally contain one or more hetero functions selected from the group consisting of —O—, —NH—, —N(C₁-C₆-alkyl)-, —N(aryl)-, —N(heteroaryl)-, —S—, —S(O)— and —S(O)₂—;
   (2) —C(O)—R₅, where R₅ is as previously defined;
   (3) —C(O)—C₁-C₆-alkyl-R₅, where R₅ is as previously defined;
   (4) —C(O)—C₂-C₆-alkenyl-R₅, where R₅ is as previously defined;
   (5) —C(O)—C₂-C₆-alkynyl-R₅, where R₅ is as previously defined;
   (6) —C₁-C₆-alkyl-M-R₅, where M=—OC(O)—, —OC(O)O—, —OC(O)NR₆—, —NR₆C(O)—, —NR₆C(O)O—, —NR₆C(O)NR₇—, —NR₆C(N)NR₇—, S(O)ₙ—, where n=0, 1 or 2, and where R₅, R₆, R₇ are as previously defined;
   (7) —C₂-C₆-alkenyl-M-R₅, where M=—OC(O)—, —OC(O)O—, —OC(O)NR₆—, —NR₆C(O)—, —NR₆C(O)O—, —NR₆C(O)NR₇—, —NR₆C(N)NR₇—, S(O)ₙ—, where n=0, 1 or 2, and where R₅, R₆, R₇ are as previously defined; and
   (8) —C₂-C₆-alkynyl-M-R₅, where M=—OC(O)—, —OC(O)O—, —OC(O)NR₆—, —NR₆C(O)—, —NR₆C(O)O—, —NR₆C(O)NR₇—, —NR₆C(N)NR₇—, S(O)ₙ—, where n=0, 1 or 2, and where R₅, R₆, R₇ are as previously defined;
R₁ is selected from the group consisting of:
   (1) hydrogen;
   (2) R₃, where R₃ is C₁-C₆-alkyl, C₂-C₆-alkenyl or C₂-C₆-alkynyl, optionally substituted with one or more substituents selected from the group consisting of:
      a. halogen;
      b. aryl;
      c. substituted-aryl;
      d. heteroaryl;
      e. substituted-heteroaryl;
      f. —O—C₁-C₆-alkyl-R₅, where R₅ is as previously defined;
      g. —NR₆R₇, where R₆ and R₇ are as previously defined.
   (3) —C(═O)—R₄, where R₄ is H or R₃ as previously defined;
   (4) —C(═O)O—R₃, where R₃ is as previously defined; and
   (5) —C(═O)N—R₆R₇, where R₆ and R₇ are as previously defined;
R₂ is selected from the group consisting of:
   (1) hydroxy protecting group;
   (2) R₅, as previously defined;
   (3) —X—Y—R₅, where X═C(O), —C(O)O—, —C(O)NH—, or absent, and Y═C₁-C₆-alkyl or absent, and R₅ is as previously defined; and
   (4) C₁-C₆-alkyl, C₂-C₆-alkenyl or C₂-C₆-alkynyl optionally substituted with one or more substituents selected from the group consisting of:
      a. halogen;
      b. aryl;
      c. substituted-aryl;
      d. heteroaryl;
      e. substituted-heteroaryl;
      f. —O—R₅ where R₅ is as previously defined;
      g. —O—C₁-C₆-alkyl-R₅ where R₅ is as previously defined;
      h. —O—C₂-C₆-alkenyl-R₅ where R₅ is as previously defined;
      i. —O—C₂-C₆-alkynyl-R₅ where R₅ is as previously defined;
      j. —NR₆R₇ where R₆ and R₇ are as previously defined; and
      k. a sugar-containing moiety;
Rp₁ is hydrogen or hydroxy protecting group; and
Z is selected from the group consisting of:
   (1) hydrogen;
   (2) hydroxy protecting groups; and
   (3) —X—R₃, where X and R₃ are as previously defined.
      A detailed description of the compounds of Formulas 5 and 6 and their synthesis is found in U.S. Pat. No. 6,673,774, incorporated herein by reference.

In yet another embodiment, the present invention provides methods of treating cystic fibrosis using the compounds of Formula 7, or a pharmaceutically acceptable salt, ester or prodrug thereof. 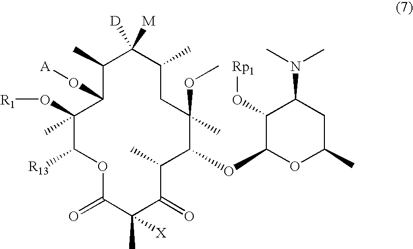
In formula 7 above,
A is selected from the group consisting of:
   (1) C₁-C₆-alkyl, C₂-C₆-alkenyl or C₂-C₆-alkynyl, optionally substituted with one or more substituents selected from the group consisting of:
      a. halogen;
      b. aryl;
      c. substituted aryl;
      d. heteroaryl;
      e. substituted heteroaryl;
      f. —O—R₅, where R₅ is selected from the group consisting of:
         i. hydrogen;
         ii. aryl;
         iii. substituted aryl;
         iv. heteroaryl; and
         v. substituted heteroaryl;
      g. —O—C₁-C₆-alkyl-R₅, where R₅ is as previously defined;
      h. —O—C₁-C₆-alkenyl-R₅, where R₅ is as previously defined
      i. —O—C₂-C₆-alkynyl-R₅, where R₅ is as previously defined; and
      j. —NR₆R₇, where R₆ and R₇ are each independently selected from hydrogen, C₁-C₆-alkyl, optionally substituted with one or more substituents selected from the group consisting of halogen, aryl, substituted aryl, heterocyclic and substituted heterocyclic, C₂-C₆-alkenyl, optionally substituted with one or more substituents selected from the group consisting of halogen, aryl, substituted aryl, heterocyclic and substituted heterocyclic, C₂-C₆-alkynyl, optionally substituted with one or more substituents selected from the group consisting of halogen, aryl, substituted aryl, heterocyclic and substituted heterocyclic or R₆R₇ taken with the nitrogen atom to which they are connected form a 3- to 7-membered ring which may optionally contain one or more hetero functions selected from the group consisting of —O—, —NH—, —N(C₁-C₆-alkyl)-, —N(aryl)-, —N(heteroaryl)-, —S—, —S(O)— and —S(O)₂—;
   (2) —C(O)—R₅, where R₅ is as previously defined;
   (3) —C(O)—C₁-C₆-alkyl-R₅, where R₅ is as previously defined;
   (4) —C(O)—C₂-C₆-alkenyl-R₅, where R₅ is as previously defined;
   (5) —C(O)—C₂-C₆-alkynyl-R₅, where R₅ is as previously defined;
   (6) —C₁-C₆-alkyl-M-R₅, where M=—OC(O)—, —OC(O)O—, —OC(O)NR₆—, —NR₆C(O)—, —NR₆C(O)O—, —NR₆C(O)NR₇—, —NR₆C(N)NR₇—, S(O)ₙ—, where n=0, 1 or 2, and where R₅, R₆, R₇ are as previously defined;
   (7) —C₂-C₆-alkenyl-M-R₅ where M=—OC(O)—, —OC(O)O—, —OC(O)NR₆—, —NR₆C(O)—, —NR₆C(O)O—, —NR₆C(O)NR₇—, —NR₆C(N)NR₇—, S(O)ₙ—, where n=0, 1 or 2, and where R₅, R₆, R₇ are as previously defined;
   (8) a sugar-containing moiety; and
   (9) —C₂-C₆-alkynyl-M-R₅ where M=—OC(O)—, —OC(O)O—, —OC(O)NR₆—, —NR₆C(O)—, —NR₆C(O)O—, —NR₆C(O)NR₇—, —NR₆C(N)NR₇—, S(O)ₙ—, where n=0, 1 or 2, and where R₅, R₆, R₇ are as previously defined;
R₁ is selected from the group consisting of:
   (1) hydrogen;
   (2) R₃, where R₃ is C₁-C₆-alkyl, C₂-C₆-alkenyl or C₂-C₆-alkynyl, optionally substituted with one or more substituents selected from the group consisting of:
      a. halogen;
      b. aryl;
      c. substituted aryl;
      d. heteroaryl;
      e. substituted heteroaryl;
      f. —O—C₁-C₆-alkyl-R₅, where R₅ is as previously defined;
      g. —NR₆R₇, where R₆ and R₇ are as previously defined.
   (3) —C(═O)—R₄, where R₄ is H or R₃ as previously defined;
   (4) —C(═O)O—R₃, where R₃ is as previously defined;
   (5) a sugar-containing moiety; and
   (6) —C(═O)N—R₆R₇, where R₆ and R₇ are as previously defined;
D and M are selected from the group consisting of:
   (1) one of D and M is hydrogen and the other is selected from the group consisting of:
      i. hydrogen;
      ii. hydroxy;
      iii. protected hydroxy; and
      iv. —NR₆R₇, where R₆ and R₇ are as previously defined; and
   (2) D and M taken together define Y, where Y is selected from the group consisting of:
      a. ═O;
      b. ═N—OH;
      c. ═N—O—R₈, where R₈ is a C₁-C₆-alkyl group, optionally substituted with a group selected from an aryl, substituted aryl, heteroaryl, or substituted heteroaryl; and
      d. ═N—O—C(R₉)(R₁₀)—O—R₁₁, where R₉ and R₁₀ are each independently selected from the group consisting of hydrogen or C₁-C₆-alkyl, and R₁₁ is selected from the group consisting of:
         i. R₈, as previously defined;
         ii. —C₁-C₆-alkyl, optionally substituted with C₁-C₆-alkoxy;
         iii. —C₁-C₆-alkyl-O—C₁-C₆-alkyl-R₅, where R₅ is as previously defined; and
         iv. a sugar-containing moiety
         v. X is selected from the group consisting of:
   (1) hydrogen;
   (2) methyl; and
   (3) halogen;
R₁₃ is selected from:
   (1) hydrogen,
   (2) C₁-C₆-alkyl optionally substituted with halogen,
   (3) C₂-C₆-alkenyl,
   (4) C₂-C₆-alkynyl,
   (5) —CH₂—R″, where R″ is selected from hydrogen, C₁-C₆-alkyl, C₂-C₆-alkenyl, C₂-C₆-alkynyl or phenyl, optionally substituted with halogen, or C₁-C₆-alkyl optionally substituted with R₅, where R₅ is as defined previously; or
   (6) —C₃-C₈-cycloalkyl;
Rp is hydrogen or hydroxy protecting group.
   A detailed description of the compounds of Formula 7 and their synthesis is found in U.S. Pat. No. 6,670,331, incorporated herein by reference.

In yet another embodiment, the present invention provides methods of treating cystic fibrosis using the compounds of Formula 8 as shown below, or a pharmaceutically acceptable salt, ester or prodrug thereof. 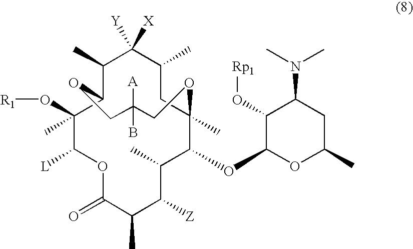
In Formula 8:
A is selected from the group consisting of:
   (a) —H;
   (b) —Rₚ, where Rₚ is a hydroxy protecting group;
   (c) —R₁, where R₁ is selected from the group consisting of:
      a. aryl;
      b. substituted aryl;
      c. heteroaryl; and
      d. substituted heteroaryl;
   (d) —OR₁, where R₁ is as previously defined;
   (e) —R₂, where R₂ is selected from the group consisting of:
      a. hydrogen;
      b. halogen;
      c. C₁-C₆ alkyl containing 0, 1, 2, or 3 heteroatoms selected from the group consisting of O, S and N, optionally substituted with one or more substituents selected from the group consisting of halogen, aryl, substituted aryl, heteroaryl, and substituted heteroaryl;
      d. C₂-C₆ alkenyl containing 0, 1, 2, or 3 heteroatoms selected from the group consisting of O, S and N, optionally substituted with one or more substituents selected the group consisting of halogen, aryl, substituted aryl, heteroaryl, and substituted heteroaryl; and
      e. C₂-C₆ alkynyl containing 0, 1, 2, or 3 heteroatoms selected from the group consisting of O, S and N, optionally substituted with one or more substituents selected from the group consisting of halogen, aryl, substituted aryl, heteroaryl, and substituted heteroaryl;
   (f) —OR₂, where R₂ is previously defined;
   (g) —S(O)ₙR₁₁, where n=0, 1 or 2, and R₁₁ is hydrogen, R₁ or R₂, where R₁ and R₂ are as previously defined
   (h) —NHC(O)R₁₁, where R₁₁ is as previously defined;
   (i) —NHC(O)NHR₁₁, where R₁₁ is as previously defined;
   (j) —NHS(O)₂R₁₁, where R₁₁ is as previously defined;
   (k) —NR₁₄R₁₅, where R₁₄ and R₁₅ are each independently R₁₁, where R₁₁ is as previously defined; and
   (l) —NHR₃, where R₃ is an amino protecting group;
B is selected from the group consisting of:
   (a) hydrogen;
   (b) deuterium;
   (c) halogen;
   (d) —OH;
   (e) —R₁, where R₁ is as previously defined;
   (f) —R₂, where R₂ is as previously defined; and
   (g) —ORₚ, where Rₚ is as previously defined;
provided that when B is halogen, —OH or ORₚ, A is R₁ or R₂;
or, alternatively, A and B taken together with the carbon atom to which they are attached are selected from the group consisting of:
   a) C═O;
   b) C(OR₂)₂, where R₂ is as previously defined;
   c) C(SR₂)₂, where R₂ is as previously defined;
   d) C[—O(CH₂)ₘ]₂, where m=2 or 3;
   e) C[—S(CH₂)ₘ]₂, where m is as previously defined,
   f) C═CHR₁₁, where R₁₁ is as previously defined;
   g) C═N—O—R₁₁, where R₁₁ is as previously defined;
   h) C═NNHR₁₁, where R₁ is as previously defined;
   i) C═NNHC(O)R₁₁, where R₁ is as previously defined;
   j) C═NNHC(O)NHR₁₁, where R₁₁ is as previously defined;
   k) C═NNHS(O)₂R₁₁, where R₁₁ is as previously defined;
   l) C═NNHR₃, where R₃ is as previously defined; and
   m) C═NR₁₁, where R₁₁ is as previously defined;
one of X and Y is hydrogen and the other is selected from the group consisting of:
   i) hydrogen;
   ii) deuterium;
   iii) —OH;
   iv) —ORₚ, where Rₚ is as previously defined; and
   v) —NR₄R₅, where R₄ and R₅ are each independently selected from the group consisting of:
      a. hydrogen; and
      b. C₁-C₁₂ alkyl, optionally substituted with one or more substituents selected from the group consisting of halogen, aryl, substituted aryl, heteroaryl and substituted heteroaryl; or
      R₄ and R₅, taken together with the nitrogen atom to which they are attached form a 3-10 membered heteroalkyl ring containing 0-2 additional hetero atoms selected from the group consisting of O, S and N; or
alternatively, X and Y taken together with the carbon atom to which they are attached are selected from the group consisting of:
   (a) C═O;
   (b) C═NR₁₁, where R₁₁ is as previously defined;
   (c) C═NC(O)R₁₁, where R₁₁ is as previously defined;
   (d) C═N—OR₆, where R₆ is selected from the group consisting of:
      a. hydrogen;
      b. —CH₂O(CH₂)₂OCH₃,
      c. —CH₂O(CH₂O)ₙCH₃, where n is as previously defined;
      d. —C₁-C₁₂ alkyl, optionally substituted with one or more substituents selected from the group consisting of aryl, substituted aryl, heteroaryl and substituted heteroaryl;
      e. C₃-C₁₂ cycloalkyl;
      f. C(O)—C₁-C₁₂ alkyl;
      g. C(O)—C₃-C₁₂ cycloalkyl;
      h. C(O)—R₁, where R₁ is as previously defined; and
      i. —Si(Rₐ)(R_{b})(R_{c}), wherein Rₐ, R_{b} and R_{c} are each independently selected from the group consisting of C₁-C₁₂ alkyl, aryl and substituted aryl; and
   (e) C═N—O—C(R₇)(R₈)—O—R₆, where R₆ is as previously defined, provided that R₆ is not C(O)—C₁-C₁₂ alkyl, C(O)—C₃-C₁₂ cycloalkyl, or C(O)—R₁; and R₇ and R₈ taken together with the carbon atom to which they are attached form a C₃ to C₁₂ cycloalkyl or each is independently selected from the group consisting of:
      a. hydrogen; and
      b. C₁-C₁₂ alkyl;
L is selected from the group consisting of:
   (a) —CH₃;
   (b) —CH₂CH₃;
   (c) —CH(OH)CH₃;
   (d) C₁-C₆ alkyl, optionally substituted with one or more substituents selected from the group consisting of aryl, substituted aryl, heteroaryl, and substituted heteroaryl;
   (e) C₂-C₆ alkenyl, optionally substituted with one or more substituents selected from the group consisting of aryl, substituted aryl, heteroaryl, and substituted heteroaryl; and
   (f) C₂-C₆ alkynyl, optionally substituted with one or more substituents selected from the group consisting of aryl, substituted aryl, heteroaryl, and substituted heteroaryl;
Z is selected from the group consisting of:
   (a) hydrogen;
   (b) —OH;
   (c) —ORₚ, where Rₚ is as previously defined;
   (d) —OR₁₁, where R₁₁ is as previously defined;
   (e) —OC(O)R₁₁, where R₁₁ is as previously defined;
   (f) —OC(O)NHR₁₁, where R₁₁ is as previously defined;
   (g) —S(O)ₙR₁₁, where n and R₁₁ are as previously defined; 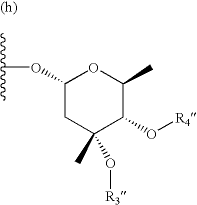
   where R₃″ is hydrogen or methyl; R₄″ is hydrogen or Rₚ, where Rₚ is as previously defined; and
R₂′ is hydrogen or Rₚ, where Rₚ, is as previously defined.
   A detailed description of the compounds of Formula 8 and their synthesis is found in U.S. Ser. No. 10/436,622, filed on May 13, 2003, incorporated herein by reference.

In another embodiment, the present invention provides methods of treating cystic fibrosis using the compounds of Formula 9, as shown below or a pharmaceutically acceptable salt, ester or prodrug thereof:

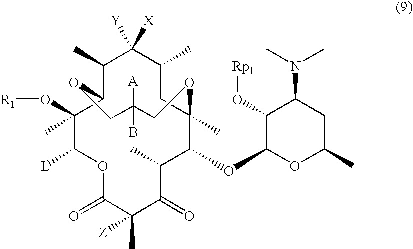

In Formula 9:
A and B are independently selected from the group consisting of hydrogen, deuterium, halogen, R₁, OR₁, S(O)ₙR₁, —NR₁C(O)R₁, —NR₁C(O)NR₃R₄, —NHS(O)ₙR₁, —CONR₃R₄, and NR₃R₄;
Each R₁ is independently selected from the group consisting of hydrogen, acyl, silane, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, a substituted or unsubstituted, saturated or unsaturated heterocyclic group;
R₃ and R₄ is independently selected from the group consisting of hydrogen, acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, saturated or unsaturated heterocyclic group; or can be taken together with the nitrogen atom to which they are attached to form a substituted or unsubstituted heterocyclic or heteroaromatic ring;
or A and B, taken together with the carbon atom to which they are attached, form a substituted or unsubstituted alicyclic, aromatic, heterocyclic or heteroaromatic ring;
or A and B, taken together with the carbon atom to which they are attached, are selected from the group consisting of CO, C═CHR₁, C═NR₁, C═NOR₁, C═NO(CH₂)ₘR₁, C═NNHR₁, C═NNHCOR₁, C═NNHCONR₁R₂, C═NNHS(O)ₙR₁, or C═N—N═CHR₁;
X and Y are independently selected from the group consisting of hydrogen, deuterium, halogen, R₁, OR₁, S(O)ₙR₁, —NR₁C(O)R₁, —NR₁C(O)NR₃R₄, —NR₁S(O)ₙR₁, —CONR₃R₄, and NR₃R₄;
or X and Y, taken together with the carbon atom to which they are attached, are selected from the group consisting of CO, C═CHR₁, C═NR₁, C═NOR₁, C═NO(CH₂)ₘR₁, C═NNHR₁, C═NNHCOR₁, C═NNHCONR₁R₂, C═NNHS(O)ₙR₁, a sugar-containing moiety or C═N—N═CHR₁;
L is selected from the group consisting of hydrogen, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, and a substituted or unsubstituted, saturated or unsaturated heterocyclic group;
W is NR₃R₄
Z is hydrogen, alkyl or halogen;
R₂′ is R₁;
m is an integer; and
n is 0, 1, or 2.

In another embodiment, the compound represented by formula 9, above, or a racemate, enantiomer, regioisomer, salt, ester or prodrug thereof is provided wherein
A is selected from:
   a) —OH;
   b) —ORₚ, where Rₚ is a hydroxy protecting group;
   c) —R₁, where R₁ is independently selected from:
      (1) aryl;
      (2) substituted aryl;
      (3) heteroaryl;
      (4) substituted heteroaryl;
      (5) heterocycloalkyl; or
      (6) substituted heterocycloalkyl;
   d) —OR₁, where R₁ is as previously defined;
   e) —R₂, where R₂ is selected from:
      (1) hydrogen;
      (2) halogen;
      (3) C₁-C₁₂ alkyl optionally containing 0, 1, 2, or 3 heteroatoms selected from O, S(O)ₙ, where n is 0, 1, or 2, or N, optionally substituted with one or more substituents selected from halogen, aryl, substituted aryl, heteroaryl, substituted heteroaryl, heterocycloalkyl, or substituted heterocycloalkyl;
      (4) C₂-C₁₂ alkenyl optionally containing 0, 1, 2, or 3 heteroatoms selected from O, S(O)ₙ, where n is as previously defined, and N, optionally substituted with one or more substituents selected from halogen, aryl, substituted aryl, heteroaryl, substituted heteroaryl, heterocycloalkyl, or substituted heterocycloalkyl; and
      (5) C₂-C₁₂ alkynyl optionally containing 0, 1, 2, or 3 heteroatoms selected from O, S(O)ₙ, where n is as previously defined, and N, optionally substituted with one or more substituents selected from halogen, aryl, substituted aryl, heteroaryl, substituted heteroaryl, heterocycloalkyl, or substituted heterocycloalkyl;
   f) —OR₂, where R₂ is independently previously defined;
   g) —S(O)ₙR₁₁, where n is as previously defined and R₁₁ is independently hydrogen, R₁ or R₂, where R₁ and R₂ are as previously defined;
   h) —NHC(O)R₁₁, where R₁₁ is as previously defined;
   i) —NHC(O)NHR₁₁, where R₁₁ is as previously defined;
   j) —NHS(O)ₙR₁₁, where n and R₁₁ are as previously defined;
   k) —NR₁₄R₁₅, where R₁₄ and R₁₅ are each independently R₁₁, where R₁₁ is as previously defined; or
   l) —NHR₃, where R₃ is an amino protecting group;
B is selected from:
   a) hydrogen;
   b) deuterium;
   c) halogen;
   d) —OH;
   e) R₁, where R₁ is as previously defined;
   f) R₂, where R₂ is as previously defined; or
   g) —ORₚ, where Rₚ is as previously defined,
   h) provided that when B is halogen, —OH, or —ORₚ, A is R₁ or R₂;
or alternatively, A and B taken together with the carbon atom to which they are attached are selected from:
   a) C(OR₁₆)(OR₁₇), where R₁₆ and R₁₇ taken together are —(CH₂)ₘ—, and where m is 2 or 3;
   b) C(SR₁₆)(SR₁₇), where R₁₆ and R₁₇ taken together are —(CH₂)ₘ and, where m is as previously defined,
   c) C═CHR₁₁, where R₁₁ is as previously defined;
   d) C═N—O—Ar₁-M-Ar₂, wherein
      (1) —Ar₁— is absent or selected from R₃₁, where R₃₁ is independently selected from:
         (a) R₁, where R₁ is as previously defined;
         (b) C₁-C₁₂ alkyl optionally containing 0, 1, 2, or 3 heteroatoms selected from O, S(O)ₙ, where n is as previously defined, and N, optionally substituted with one or more substituents selected from halogen, aryl, substituted aryl, heteroaryl, substituted heteroaryl, heterocycloalkyl, or substituted heterocycloalkyl;
         (c) C₂-C₁₂ alkenyl optionally containing 0, 1, 2, or 3 heteroatoms selected from O, S(O)ₙ, where n is as previously defined, and N, optionally substituted with one or more substituents selected from halogen, aryl, substituted aryl, heteroaryl, substituted heteroaryl, heterocycloalkyl, or substituted heterocycloalkyl; or
         (d) C₂-C₁₂ alkynyl optionally containing 0, 1, 2, or 3 heteroatoms selected from O, S(O)ₙ, where n is as previously defined, and N, optionally substituted with one or more substituents selected from halogen, aryl, substituted aryl, heteroaryl, substituted heteroaryl, heterocycloalkyl, or substituted heterocycloalkyl;
      (2) -M- is absent or selected from:
         (a) —C₁-C₁₂ alkyl optionally containing:
            (1) 0-3 heteroatoms selected from O, S(O)ₙ, where n is as previously defined, or N; and
            (2) 0-3 groups selected from —C═N—, —N═N, —C(O)—;
         (b) —C₂-C₁₂ alkenyl optionally containing:
            (1) 0-3 heteroatoms selected from O, S(O)ₙ, where n is as previously defined, or N; and
            (2) 0-3 groups selected from —C═N—, —N═N, —C(O)—;
         (c) —C₂-C₁₂ alkynyl optionally containing;
            (1) 0-3 heteroatoms selected from O, S(O)ₙ, where n is as previously defined, or N; and
            (2) 0-3 groups selected from —C═N—, —N═N, —C(O)—;
         (d) substituted aryl;
         (e) substituted heteroaryl;
         (f) heterocycloalkyl; or
         (g) substituted heterocycloalkyl; and
      (3) —Ar₂ is absent or selected from:
         (a) aryl;
         (b) substituted aryl;
         (c) heteroaryl;
         (d) substituted heteroaryl;
         (e) heterocycloalkyl; or
         (f) substituted heterocycloalkyl;
   e) C═NNHR₁₁, where R₁₁ is as previously defined;
   f) C═NNHC(O)R₁₁, where R₁₁ is as previously defined;
   g) C═NNHC(O)NHR₁₁, where R₁₁ is as previously defined;
   h) C═NNHS(O)ₙR₁₁, where n and R₁ are as previously defined;
   i) C═NNHR₃, where R₃ is as previously defined;
   j) C═NR₁₁, where R₁₁ is as previously defined; or
   k) C═N—N═CHR₁₁, where R₁₁ is as previously defined;
one of X and Y is hydrogen and the other is selected from:
   a) hydrogen;
   b) deuterium;
   c) —OH;
   d) —ORₚ, where Rₚ is as previously defined;
   e) —NR₄R₅, where R₄ and R₅ are each independently selected from:
      (1) hydrogen;
      (2) C₁-C₁₂ alkyl, optionally substituted with one or more substituents selected from halogen, aryl, substituted aryl, heteroaryl, substituted heteroaryl, heterocycloalkyl, or substituted heterocycloalkyl; or
      (3) R₄ and R₅, taken together with the nitrogen atom to which they are attached to form a heterocycloalkyl moiety;
alternatively, X and Y taken together with the carbon atom to which they are attached are selected from:
   a) C═O;
   b) C═N—Q, wherein Q is selected from:
      (1) —R₁₁, where R₁₁ is as previously defined;
      (2) amino protecting group;
      (3) —C(O)R₁₁, where R₁₁ is as previously defined;
      (4) —OR6, where R₆ is independently selected from:
         a. hydrogen;
         b. —CH₂O(CH₂)₂OCH₃,
         c. —CH₂O(CH₂O)ₙCH₃, where n is as previously defined;
         d. —C₁-C₁₂ alkyl, optionally substituted with one or more substituents selected from aryl, substituted aryl, heteroaryl, substituted heteroaryl, heterocycloalkyl, or substituted heterocycloalkyl;
         e. —C₃-C₁₂ cycloalkyl;
         f. —C(O)—C₁-C₁₂ alkyl;
         g. —C(O)—C₃-C₁₂ cycloalkyl;
         h. —C(O)—R₁, where R₁ is as previously defined; or
         i. —Si(Rₐ)(R_{b})(R_{c}), wherein Rₐ, R_{b} and R_{c} are each independently selected from C₁-C₁₂ alkyl, aryl or substituted aryl; or
      (5) O—C(R₇)(R₈)—O—R₆, where R₆ is as previously defined, provided that R₆ is not C(O)—C₁-C₁₂ alkyl, C(O)—C₃-C₁₂ cycloalkyl, or C(O)—R₁, and R₇ and R₈ taken together with the carbon atom to which they are attached form a C₃-C₁₂ cycloalkyl group or each independently is selected from:
         a. hydrogen; or
         b. C₁-C₁₂ alkyl;
L is selected from:
   a) —CH₃;
   b) —CH₂CH₃;
   c) —CH(OH)CH₃;
   d) —(CH₂)ₙNHC(O)—R₁ ₁, wherein n and R₁₁ are as previously defined;
   e) C₁-C₆ alkyl, optionally substituted with one or more substituents selected from aryl, substituted aryl, heteroaryl, substituted heteroaryl, heterocycloalkyl, or substituted heterocycloalkyl;
   f) C₂-C₆ alkenyl, optionally substituted with one or more substituents selected from aryl, substituted aryl, heteroaryl, substituted heteroaryl, heterocycloalkyl, or substituted heterocycloalkyl; or
   g) C₂-C₆ alkynyl, optionally substituted with one or more substituents selected from aryl, substituted aryl, heteroaryl, substituted heteroaryl, heterocycloalkyl, or substituted heterocycloalkyl;
W is —NR₂₀R₂₁, where R₂₀ and R₂₁ are each independently selected from:
   a) hydrogen;
   b) C₁-C₁₂ alkyl, optionally substituted with one or more substituents selected from halogen, aryl, substituted aryl, heteroaryl, substituted heteroaryl, heterocycloalkyl, or substituted heterocycloalkyl;
   c) C₂-C₁₂ alkenyl, optionally substituted with one or more substituents selected from halogen, aryl, substituted aryl, heteroaryl, substituted heteroaryl, heterocycloalkyl, or substituted heterocycloalkyl;
   d) C₂-C₁₂ alkynyl, optionally substituted with one or more substituents selected from halogen, aryl, substituted aryl, heteroaryl, substituted heteroaryl, heterocycloalkyl, or substituted heterocycloalkyl; or
   e) R₂₀ and R₂₁, taken together with the nitrogen atom to which they are attached form a heterocycloalkyl moiety; or
Z is selected from:
   a) hydrogen;
   b) methyl; or
   c) halogen; and
R₂′ is hydrogen or Rₚ, where Rₚ, is as previously defined.

A detailed description of the compound of Formula 9 and their synthesis is found in U.S. Ser. No. 10/717,290, filed Nov. 19, 2003, U.S. Ser. No. 10/758,409, filed Jan. 14, 2004, and U.S. Ser. No. 10/763,377, filed Jan. 23, 2004, all incorporated herein by reference.

In another embodiment, the present invention provides methods of treating cystic fibrosis using the compounds of Formula 10 as shown below, or a pharmaceutically acceptable salt, ester or prodrug thereof. 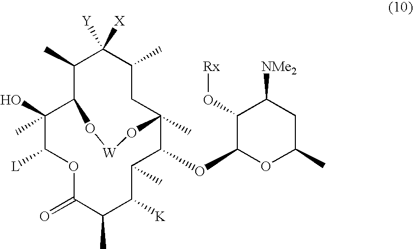
In Formula 10:
W is selected from the group consisting of:
   (a) —CH₂—C(A)═C(B)—CH₂—;
      wherein,
      A and B are independently selected from the group consisting of:
         (i) hydrogen;
         (ii) deuterium;
         (iii) halogen;
         (iv) R₁, wherein R₁ is selected from the group consisting of:
            a. C₁-C₆ alkyl, optionally substituted with one or more substituents selected from the group consisting of: halogen, aryl, substituted aryl, heteroaryl, and substituted heteroaryl;
            b. C₂-C₆ alkenyl, optionally substituted with one or more substituents selected from the group consisting of: halogen, aryl, substituted aryl, heteroaryl, and substituted heteroaryl; and
            c. C₂-C₆ alkynyl, optionally substituted with one or more substituents selected from the group consisting of: halogen, aryl, substituted aryl, heteroaryl, and substituted heteroaryl;
         (ix) R₂, wherein R₂ is selected from the group consisting of:
            a. aryl;
            b. heteroaryl;
            c. substituted aryl; and
            d. substituted heteroaryl;
         (x) —(C₁-C₃-alkyl)-M-(C₁-C₃-alkyl)-R₂, wherein M=—O—, —NH—, —N(CH₃)—, —NHC(O)—, —S(O)ₙ—, wherein n=0, 1 or 2, and R₂ is as previously defined;
         (xi) —(C₁-C₃-alkyl)-M-R₂, wherein M and R₂ are as previously defined;
         (xii) —C(O)-J-R₃, wherein J is absent, O or S, and R₃ is H, R₁ or R₂, where R₁ and R₂ are as previously defined; and
         (xiii) —C(O)—NR₁₁R₁₂, wherein R₁₁ and R₁₂ are each independently selected from the group consisting of:
            a. hydrogen;
            b. C₁-C₆-alkyl, optionally substituted with one or more substituents selected from the group consisting of: halogen, aryl, substituted aryl, heteroaryl, and substituted heteroaryl;
            c. C₂-C₆-alkenyl, optionally substituted with one or more substituents selected from the group consisting of: halogen, aryl, substituted aryl, heteroaryl, and substituted heteroaryl;
            d. C₂-C₆-alkynyl, optionally substituted with one or more substituents selected from the group consisting of: halogen, aryl, substituted aryl, heteroaryl, and substituted heteroaryl; and
            e. R₁₁ and R₁₂ taken together with the nitrogen atom to which they are connected form a 3- to 7-membered ring which may optionally contain one or more heterofunctions selected from the group consisting of: —O—, —NH—, —N(C₁-C₆-alkyl)-, —N(R₂)—, —S(O)ₙ—, wherein n and R₂ are as previously defined;
   (b) —CH₂—CH(A)-C(B)═CH—, wherein A and B are as previously defined;
   (c) —CH₂—CH(E)-CH(G)-CH₂—;
      wherein E and G are independently selected from the group consisting of:
         (i) A, wherein A is as previously defined;
         (ii) —OH;
         (iii) —OR^{p}, wherein R^{p} is a hydroxy protecting group;
         (iv) —O—R₉, wherein R₉ is R₁ or R₂, and wherein R₁ and R₂ are as previously defined;
         (v) —S(O)ₙR₉, wherein n and R₉ are as previously defined;
         (vi) —NHC(O)R₃, wherein R₃ is as previously defined;
         (vii) —NHC(O)NR₁₁R₃, wherein R₁₁ and R₃ are as previously defined;
         (viii) —NHS(O)₂R₉, wherein R₉ is as previously defined;
         (ix) —NHR₁₃, wherein R₁₃ is an amino protecting group; and
         (x) —NR₁₁R₁₂, wherein R₁₁ and R₁₂ are as previously defined; 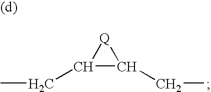
      wherein:
         (i) -Q- is selected from the group consisting of: —O—; —O—C(O)—CH(R₇)—; —N(R₇)—; —O—C(O)—N(R₇)—; —O—C(O)—O—; —N(R₇)—N═N—;
   —C(R₇)═N—O—; and —CH(R₇)—N(R₈)—O—; wherein R₇ and R₈ are independently selected from R₃, wherein R₃ is as previously defined; or
      (ii) -Q- taken together with the two carbon atoms it is attached to is selected from the group consisting of:
         a. cycloalkylene;
         b. cycloalkenylene; and
         c. heterocycloalkylene; and
   (e) —CH₂—C(R₄)(R₅)—CH₂—CH₂—;
      wherein R₄ and R₅ taken together with the carbon atom to which they are attached are selected from the group consisting of:
         (i) C═O;
         (ii) C(OR₁)₂, wherein R₁ is as previously defined;
         (iii) C(SR₁)₂, wherein R₁ is as previously defined;
         (iv) C[—O(CH₂)ₘ]₂, wherein m is 2 or 3;
         (v) C[—S(CH₂)ₘ]₂, wherein m is as previously defined,
         (vi) C═CHR₃, wherein R₃ is as previously defined;
         (vii) C═N—O—R₃, wherein R₃ is as previously defined;
         (viii) C═NNHR₃, wherein R₃ is as previously defined;
         (ix) C═NNHC(O)R₃, wherein R₃ is as previously defined;
         (x) C═NNHC(O)NR₁₁R₃, wherein R₁₁ and R₃ are as previously defined;
         (xi) C═NNHS(O)₂R₉, wherein R₉ is as previously defined;
         (xii) C═NNHR₁₃, wherein R₁₃ is as previously defined; and
         (xiii) C═NR₉, wherein R₉ is as previously defined;
X and Y are:
   (a) independently selected from the group consisting of:
      i) hydrogen;
      ii) deuterium;
      i) —OH;
      ii) —OR^{p}, wherein R^{p} is as previously defined; and
      iii) —NR₁₄R₁₅, wherein R₁₄ and R₁₅ are each independently selected from the group consisting of:
         a. hydrogen;
         b. C₁-C₁₂ alkyl, optionally substituted with one or more substituents selected from the group consisting of halogen, aryl, substituted aryl, heteroaryl and substituted heteroaryl; and
         c. R₁₄ and R₁₅, taken together with the nitrogen atom to which they are attached form a 3 to 10 membered heterocycloalkyl ring optionally substituted with one or more hetero atoms selected from the group consisting of O, S and N; or
   (b) taken together with the carbon atom to which they are attached are selected from the group consisting of:
      (i) C═O;
      (ii) C═NR₃, wherein R₃ is as previously defined;
      (iii) C═NC(O)R₃, wherein R₃ is as previously defined;
      (iv) C═N—OR₆, wherein R₆ is selected from the group consisting of:
         a. hydrogen;
         b. —CH₂O(CH₂)₂OCH₃;
         c. —CH₂O(CH₂O)ₙCH₃, wherein n is as previously defined;
         d. C₁-C₁₂ alkyl, optionally substituted with one or more substituents selected from the group consisting of halogen, aryl, substituted aryl, heteroaryl and substituted heteroaryl;
         e. C₃-C₁₂ cycloalkyl;
         f. C(O)—C₁-C₁₂ alkyl;
         g. C(O)—(C₃-C₁₂ cycloalkyl);
         h. C(O)—R₂, wherein R₂ is as previously defined; and
         i. —Si(Rₐ)(R_{b})(R_{c}), wherein Rₐ, R_{b} and R_{c} are each independently selected from the group consisting of C₁-C₁₂ alkyl, aryl and substituted aryl; and
      (v) C═N—O—C(R₁₆)(R₇)—O—R₁₈, wherein R₁₆ and R₁₇ taken together with the carbon atom to which they are attached form a C₃ to C₁₂ cycloalkyl group or each independently is selected from the group consisting of: hydrogen, and C₁-C₁₂ alkyl; and R₁₈ is selected from the group consisting of:
         a. hydrogen;
         b. —CH₂O(CH₂)₂OCH₃;
         c. —CH₂O(CH₂O)ₙCH₃, wherein n is as previously defined;
         d. C₁-C₁₂ alkyl, optionally substituted with one or more substituents selected from the group consisting of halogen, aryl, substituted aryl, heteroaryl and substituted heteroaryl;
         e. C₃-C₁₂ cycloalkyl; and
         f. —Si(Rₐ)(R_{b})(R_{c}), wherein Rₐ, R_{b} and R_{c} are as previously defined;
L is selected from the group consisting of:
   (a) —CH(OH)CH₃;
   (b) C₁-C₆ alkyl, optionally substituted with one or more substituents selected from the group consisting of aryl, substituted aryl, heteroaryl, and substituted heteroaryl;
   (c) C₂-C₆ alkenyl, optionally substituted with one or more substituents selected from the group consisting of aryl, substituted aryl, heteroaryl, and substituted heteroaryl; and
   (d) C₂-C₆ alkynyl, optionally substituted with one or more substituents selected from the group consisting of aryl, substituted aryl, heteroaryl, and substituted heteroaryl;
K is selected from the group consisting of:
   (a) R₁₀, wherein R₁₀ is selected from the group consisting of:
      (i) hydrogen;
      (ii) —OR^{p}, wherein R^{p} is as previously defined;
      (iii) —OR₃, wherein R₃ is as previously defined;
      (iv) —OC(O)R₃, wherein R₃ is as previously defined;
      (v) —OC(O)NR₁₁R₃, wherein R₁ and R₃ are as previously defined; and
      (vi) —S(O)ₙR₉, wherein n and R₉ are as previously defined; and 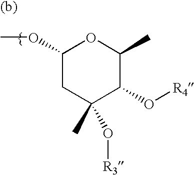
   wherein R₃″ is hydrogen or methyl; R₄″ is hydrogen or R^{p}, wherein R^{p} is as previously defined; and
Rₓ is hydrogen or R^{p}, wherein R^{p} is as previously defined.
   A detailed description of the compounds of Formula 10 and their synthesis is found in U.S. Pat. No. 6,753,318, U.S. Ser. No. 10/758,409, filed Jan. 14, 2004, and U.S. Ser. No. 10/763,377, filed Jan. 23, 2004, all incorporated herein by reference.

In another embodiment, the present invention provides methods of treating cystic fibrosis using the compounds of Formula 11, as shown below or a pharmaceutically acceptable salt, ester or prodrug thereof. 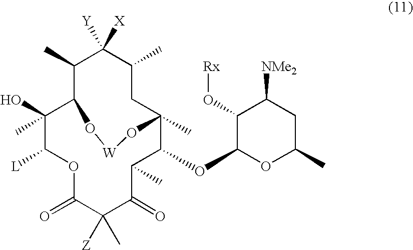
In Formula 11:
W is selected from the group consisting of:
   (a) —CH₂—C(A)═C(B)—CH₂—;
      wherein,
      A and B are independently selected from the group consisting of:
         (i) hydrogen;
         (ii) deuterium;
         (iii) halogen;
         (iv) R₁, wherein R₁ is selected from the group consisting of:
            a. C₁-C₆ alkyl, optionally substituted with one or more substituents selected from the group consisting of: halogen, aryl, substituted aryl, heteroaryl, and substituted heteroaryl;
            b. C₂-C₆ alkenyl, optionally substituted with one or more substituents selected from the group consisting of: halogen, aryl, substituted aryl, heteroaryl, and substituted heteroaryl; and
            c. C₂-C₆ alkynyl, optionally substituted with one or more substituents selected from the group consisting of: halogen, aryl, substituted aryl, heteroaryl, and substituted heteroaryl;
         (v) R₂, wherein R₂ is selected from the group consisting of:
            a. aryl;
            b. heteroaryl;
            c. substituted aryl; and
            d. substituted heteroaryl;
         (vi) —(C₁-C₃-alkyl)-M-(C₁-C₃-alkyl)-R₂, wherein M=—O—, —NH—, —N(CH₃)—, —NHC(O)— or —S(O)ₙ—, wherein n=0, 1 or 2, and R₂ is as previously defined;
         (vii) —(C₁-C₃-alkyl)-M-R₂, wherein M and R₂ are as previously defined;
         (viii) —C(O)-J-R₃, wherein J is absent, O or S, and R₃ is H, R₁ or R₂; where R₁ and R₂ are as previously defined, and
         (ix) —C(O)—NR₁₁R₁₂, wherein R₁₁ and R₁₂ are each independently selected from the group consisting of:
            a. hydrogen;
            b. C₁-C₆-alkyl, optionally substituted with one or more substituents selected from the group consisting of: halogen, aryl, substituted aryl, heteroaryl, and substituted heteroaryl;
            c. C₂-C₆-alkenyl, optionally substituted with one or more substituents selected from the group consisting of: halogen, aryl, substituted aryl, heteroaryl, and substituted heteroaryl;
            d. C₂-C₆-alkynyl, optionally substituted with one or more substituents selected from the group consisting of: halogen, aryl, substituted aryl, heteroaryl, and substituted heteroaryl; and
            e. R₁₁ and R₁₂ taken together with the nitrogen atom to which they are connected form a 3- to 7-membered ring which may optionally contain one or more heterofunctions selected from the group consisting of: —O—, —NH—, —N(C₁-C₆-alkyl)-, —N(R₂)—, —S(O)ₙ—, wherein n and R₂ are as previously defined;
   (b) —CH₂—CH(A)-C(B)═CH—, wherein A and B are as previously defined;
   (c) —CH₂—CH(E)-CH(G)-CH₂—;
      wherein E and G are independently selected from the group consisting of:
         (i) A, wherein A is as previously defined;
         (ii) —OH;
         (iii) —OR^{p}, wherein R^{p} is a hydroxy protecting group;
         (iv) —O—R₉, wherein R₉ is R₁ or R₂, and wherein R₁ and R₂ are as previously defined;
         (v) —S(O)ₙR₉, wherein n and R₉ are as previously defined;
         (vi) —NHC(O)R₃, wherein R₃ is as previously defined;
         (vii) —NHC(O)NR₁₁R₃, wherein R₁₁ and R₃ are as previously defined;
         (viii) —NHS(O)₂R₉, wherein R₉ is as previously defined;
         (ix) —NHR₁₃, wherein R₁₃ is an amino protecting group; and
         (x) —NR₁₁R₁₂, wherein R₁₁ and R₁₂ are as previously defined; 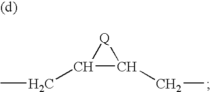
      wherein:
         (i) -Q- is selected from the group consisting of: —O—; —O—C(O)—CH(R₇)—; —N(R₇)—; —O—C(O)—N(R₇)—; —O—C(O)—O—; —N(R₇)—N═N—; —C(R₇)═N—O—; and —CH(R₇)—N(R₈)—O—; wherein R₇ and R₈ are independently selected from R₃, wherein R₃ is as previously defined; or
         (ii) -Q- taken together with the two carbon atoms it is attached to is selected from the group consisting of:
            a. cycloalkylene;
            b. cycloalkenylene; and
            c. heterocycloalkylene; and
   (e) —CH₂—C(R₄)(R₅)—CH₂—CH₂—;
      wherein R₄ and R₅ taken together with the carbon atom to which they are attached are selected from the group consisting of:
         (i) C═O;
         (ii) C(OR₁)₂, wherein R₁ is as previously defined;
         (iii) C(SR₁)₂, wherein R₁ is as previously defined;
         (iv) C[—O(CH₂)ₘ]₂, wherein m is 2 or 3;
         (v) C[—S(CH₂)ₘ]₂, wherein m is as previously defined,
         (vi) C═CHR₃, wherein R₃ is as previously defined;
         (vii) C═N—O—R₃, wherein R₃ is as previously defined;
         (viii) C═NNHR₃, wherein R₃ is as previously defined;
         (ix) C═NNHC(O)R₃, wherein R₃ is as previously defined;
         (x) C═NNHC(O)NR₁₁R₃, wherein R₁₁ and R₃ are as previously defined;
         (xi) C═NNHS(O)₂R₉, wherein R₉ is as previously defined;
         (xii) C═NNHR₁₃, wherein R₁₃ is as previously defined; and
         (xiii) C═NR₉, wherein R₉ is as previously defined;
X and Y are:
   (a) independently selected from the group consisting of:
      i) hydrogen;
      ii) deuterium;
      iii) —OH;
      iv) —OR^{p}, wherein R^{p} is as previously defined; and
      v) —NR₁₄R₁₅, wherein R₁₄ and R₁₅ are each independently selected from the group consisting of:
         a. hydrogen;
         b. C₁-C₁₂ alkyl, optionally substituted with one or more substituents selected from the group consisting of halogen, aryl, substituted aryl, heteroaryl and substituted heteroaryl; and
         c. R₁₄ and R₁₅, taken together with the nitrogen atom to which they are attached form a 3 to 10 membered heterocycloalkyl ring optionally substituted with one or more hetero atoms selected from the group consisting of O, S and N; or
   (b) taken together with the carbon atom to which they are attached are selected from the group consisting of:
      (i) C═O;
      (ii) C═NR₃, wherein R₃ is as previously defined;
      (iii) C═NC(O)R₃, wherein R₃ is as previously defined;
      (iv) C═N—OR₆, wherein R₆ is selected from the group consisting of:
         a. hydrogen;
         b. —CH₂O(CH₂)₂OCH₃;
         c. —CH₂O(CH₂O)ₙCH₃, wherein n is as previously defined;
         d. C₁-C₁₂ alkyl, optionally substituted with one or more substituents selected from the group consisting of halogen, aryl, substituted aryl, heteroaryl and substituted heteroaryl;
         e. C₃-C₁₂ cycloalkyl;
         f. C(O)—C₁-C₁₂ alkyl;
         g. C(O)—(C₃-C₁₂ cycloalkyl);
         h. C(O)—R₂, wherein R₂ is as previously defined; and
         i. —Si(Rₐ)(R_{b})(R_{c}), wherein Rₐ, R_{b} and R_{c} are each independently selected from the group consisting of C₁-C₁₂ alkyl, aryl and substituted aryl;
      (v) C═N—O—C(R₁₆)(R₁₇)—O—R₁₈, wherein R₁₆ and R₁₇ taken together with the carbon atom to which they are attached form a C₃ to C₁₂ cycloalkyl group or each independently is selected from the group consisting of: hydrogen and C₁-C₁₂ alkyl; and R₁₈ is selected from the group consisting of:
         a. hydrogen;
         b. —CH₂O(CH₂)₂OCH₃;
         c. —CH₂O(CH₂O)ₙCH₃, wherein n is as previously defined;
         d. C₁-C₁₂ alkyl, optionally substituted with one or more substituents selected from the group consisting of halogen, aryl, substituted aryl, heteroaryl and substituted heteroaryl;
         e. C₃-C₁₂ cycloalkyl; and
         f. —Si(Rₐ)(R_{b})(R_{c}), wherein Rₐ, R_{b} and R_{c} are as previously defined;
L is selected from the group consisting of:
   (a) —CH(OH)CH₃;
   (b) C₁-C₆ alkyl, optionally substituted with one or more substituents selected from the group consisting of aryl, substituted aryl, heteroaryl, and substituted heteroaryl;
   (c) C₂-C₆ alkenyl, optionally substituted with one or more substituents selected from the group consisting of aryl, substituted aryl, heteroaryl, and substituted heteroaryl; and
   (d) C₂-C₆ alkynyl, optionally substituted with one or more substituents selected from the group consisting of aryl, substituted aryl, heteroaryl, and substituted heteroaryl;
Z is selected from the group consisting of:
   (a) hydrogen;
   (b) methyl; and
   (c) halogen; and
Rₓ is hydrogen or R^{p}, wherein R^{p} is as previously defined.
   A detailed description of the compounds of Formula 11 and their synthesis is found in U.S. Ser. No. 10/205,018, filed Jul. 25, 2004, U.S. Ser. No. 10/758,409, filed Jan. 14, 2004, and U.S. Ser. No. 10/763,377, filed Jan. 23, 2004, incorporated herein by reference.

In another embodiment, the present invention provides methods of treating cystic fibrosis using the compounds of Formulas 12-15 as shown below, or a pharmaceutically acceptable salt, ester or prodrug thereof. 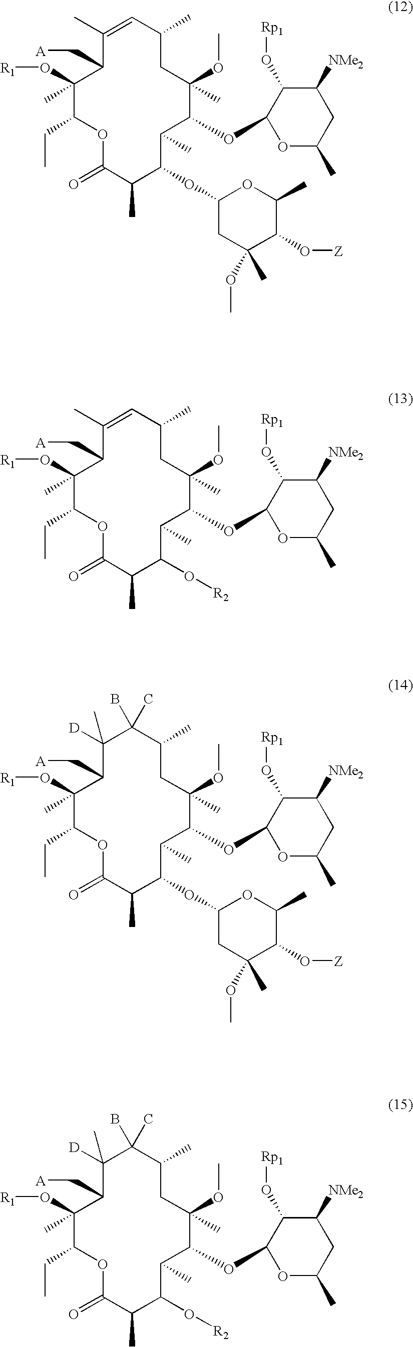
In formulas 12-15 above:
A is selected from the group consisting of
   (1) C₁-C₆-alkyl, C₂-C₆-alkenyl or C₂-C₆-alkynyl, optionally substituted with one or more substituents selected from the group consisting of:
      i. halogen;
      ii. aryl;
      iii. substituted aryl;
      iv. heterocyclic;
      v. substituted heterocyclic;
      vi. —O—R₅, where R₅ is selected from the group consisting of:
         a. hydrogen;
         b. aryl;
         c. substituted aryl;
         d. heterocyclic; and
         e. substituted heterocyclic;
      vii. —O—C₁-C₆-alkyl-R₅, where R₅ is as previously defined;
      viii. —O—C₂-C₆-alkenyl-R₅, where R₅ is as previously defined;
      ix. —O—C₂-C₆-alkynyl-R₅, where R₅ is as previously defined; and
      x. —NR₆R₇, where R₆ and R₇ are each independently selected from the group consisting of:
         a. hydrogen;
         b. C₁-C₆-alkyl, optionally substituted with one or more substituents selected from the group consisting of:
            (I) halogen;
            (II) aryl;
            (III) substituted aryl;
            (IV) heterocyclic; and
            (V) substituted heterocyclic;
         c. C₂-C₆-alkenyl, optionally substituted with one or more substituents selected from the group consisting of:
            (I) halogen;
            (II) aryl;
            (III) substituted aryl;
            (IV) heterocyclic; and
            (V) substituted heterocyclic;
         d. C₂-C₆-alkynyl, optionally substituted with one or more substituents selected from the group consisting of:
            (I) halogen;
            (II) aryl;
            (III) substituted aryl;
            (IV) heterocyclic; and
            (V) substituted heterocyclic, or
         e. R₆ R₇ taken with the nitrogen atom to which they are connected form a 3- to 7-membered ring which may optionally contain one or more functions selected from the group consisting of:
            (I) —O—;
            (II) —NH—;
            (III) —N(C₁-C₆-alkyl)-;
            (IV)—N(aryl);
            (V)—N(heteroaryl);
            (VI) —S—;
            (VII)—S(O);
            (VIII)—S(O)₂—; and
            (IX)—C(O);
   (2) —C(O)—R₅, where R₅ is as previously defined;
   (3) —C(O)—C₁-C₆-alkyl-R₅, where R₅ is as previously defined;
   (4) —C(O)—C₂-C₆-alkenyl-R₅, where R₅ is as previously defined;
   (5) —C(O)—C₂-C₆-alkynyl-R₅, where R₅ is as previously defined;
   (6) C₁-C₆-alkyl-M-R₅, where M is —OC(O)—, —OC(O)O—, —OC(O)NR₆—, —C(O)NR₆, —NR₆C(O)—, —NR₆C(O)O—, —NR₆C(O)NR₇—, —NR₆C(NH)NR₇— or S(O)ₙ—, where n=0, 1 or 2, and where R₅, R₆, R₇ are as previously defined;
   (7) —C₂-C₆-alkenyl-M-R₅, where M and R₅ are as previously defined; and
   (8) —C₂-C₆-alkynyl-M-R₅, where M and R₅ are as previously defined;
B, C, and D may be present singly or in combination and are independently selected from the group consisting of:
   (1) hydrogen;
   (2) halogen;
   (3) C₁-C₆-alkyl;
   (4) aryl;
   (5) substituted aryl;
   (6) heteroaryl;
   (7) substituted heteroaryl;
   (8) O—R₅ where R₅ is as previously defined;
   (9) B and C taken together are ═O;
   (10) B and C taken together are ═N—O—R₅, where R₅ is as previously defined;
   (11) B and C taken together are ═N—N—R₆R₇, where R₆ and R₇ are as previously defined;
   (12) B and D taken together are —O—;
   (13) B and D taken together are —S—;
   (14) B and D taken together are —N(R₆)—, where R₆ is as previously defined; and
   (15) B and D taken together with the carbon atoms to which they are attached form a 4- to 8-membered ring which may optionally contain one or more functions selected from the group consisting of:
      i. —O—;
      ii. —NH—;
      iii. —N(C₁-C₆-alkyl)-;
      iv. —N(aryl)-;
      v. —N(heteroaryl)-;
      vi. —S—;
      vii. —S(O)—;
      viii. —S(O)₂—; and
      ix. —C(O)—;
R₁ is selected from the group consisting of:
   (1) hydrogen;
   (2) R₃, where R₃ is C₁-C₆-alkyl, C₂-C₆-alkenyl or C₂-C₆-alkynyl, optionally substituted with one or more substituents selected from the group consisting of:
      i. halogen;
      ii. aryl;
      iii. substituted-aryl;
      iv. heteroaryl;
      v. substituted-heteroaryl;
      vi. —O—C₁-C₆-alkyl-R₅, where R₅ is as previously defined; and
      vii. —N—R₆R₇, where R₆ and R₇ are as previously defined;
   (3) —C(O)—R₄, where R₄ is hydrogen or R₃, where R₃ is as previously defined;
   (4) —C(O)O—R₃, where R₃ is as previously defined; and
   (5) —C(O)N—R₆R₇, where R₆ and R₇ are as previously defined;
R₂ is selected from the group consisting of:
   (1) a hydroxy protecting group;
   (2) R₅, where R₅ is as previously defined;
   (3) —X—Y—R₅, where X is —C(O), —C(O)O—, —C(O)NR₆—, or absent, and Y is C₁-C₆-alkyl or absent, where R₅ and R₆ are as previously defined; and
   (4) C₁-C₆-alkyl, C₂-C₆-alkenyl or C₂-C₆-alkynyl optionally substituted with one or more substituents selected from the group consisting of:
      i. halogen;
      ii. aryl;
      iii. substituted-aryl;
      iv. heteroaryl;
      v. substituted-heteroaryl;
      vi. —O—R₅ where R₅, is as previously defined;
      vii. —O—C₁-C₆-alkyl-R₅, where R₅ is as previously defined;
      viii. —O—C₂-C₆-alkenyl-R₅, where R₅ is as previously defined;
      ix. —O—C₂-C₆-alkynyl-R₅, where R₅ is as previously defined; and
      x. —N—R₆R₇, where R₆ and R₇ are as previously defined;
Rp₁ is hydrogen or a hydroxy protecting group; and
Z is selected from the group consisting of:
   (1) hydrogen;
   (2) a hydroxy protecting group; and
   (3) —X—R₃, where X and R₃ are as previously defined.
      A detailed description of the compounds of Formulas 12-15 and their synthesis is found in U.S. Ser. No. 10/178,991, filed on Jun. 25, 2002, U.S. Pat. No. 6,812,216, incorporated herein by reference.

In yet another embodiment, the present invention provides methods of treating cystic fibrosis using the compounds of Formula 16, as shown below or a pharmaceutically acceptable salt, ester or prodrug thereof. 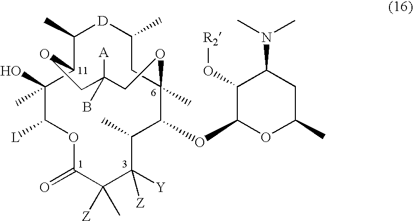
In Formula 16:
A is
   i) —OH;
   ii) —ORₚ, where Rₚ is a hydroxy protecting group;
   iii) —R₁, where R₁ is aryl, substituted aryl, heteroaryl, or substituted heteroaryl;
   iv) —ORₚ, where R₁ is as previously defined;
   v) —R₂, where R₂ is
      (a) hydrogen;
      (b) halogen;
      (c) —C₁-C₆ alkyl containing 0, 1, 2, or 3 heteroatoms selected from O, S or N, optionally substituted with one or more substituents selected from halogen, aryl, substituted aryl, heteroaryl, or substituted heteroaryl;
      (d) —C₂-C₆ alkenyl containing 0, 1, 2, or 3 heteroatoms selected from O, S, or N, optionally substituted with one or more substituents selected from halogen, aryl, substituted aryl, heteroaryl, or substituted heteroaryl; or
      (e) —C₂-C₆ alkynyl containing 0, 1, 2, or 3 heteroatoms selected from O, S or N, optionally substituted with one or more substituents selected from halogen, aryl, substituted aryl, heteroaryl, or substituted heteroaryl;
   vi) —OR₂, where R₂ is previously defined;
   vii) —S(O)ₙR₁₁, where n=0, 1 or 2, and R₁₁ is R₁ or R₂, where R₁ and R₂ are as previously defined;
   viii) —NHC(O)R₁₁, where R₁₁ is as previously defined;
   ix) —NHC(O)NHR₁₁, where R₁ is as previously defined;
   x) —NHS(O)₂R₁₁, where R₁₁ is as previously defined;
   xi) —NR₁₄R₁₅, where R₁₄ and R₁₅ are each independently R₁₁ where R₁₁ is as previously defined; or
   xii) —NHR₃, where R₃ is an amino protecting group;
B is
   i) hydrogen;
   ii) deuterium;
   iii) halogen;
   iv) —OH;
   v) —R₁, where R₁ is as previously defined;
   vi) —R₂, where R₂ is as previously defined; or
   vii) —ORₚ, where Rₚ is as previously defined, provided that when B is halogen, —OH or ORₚ, A is R₁ or R₂, where R₁ and R₂ are previously defined;
or, alternatively, A and B taken together with the carbon atom to which they are attached are
   i) C═O;
   ii) C(OR₂)₂, where R₂ is as previously defined;
   iii) C(SR₂)₂, where R₂ is as previously defined;
   iv) C[—O(CH₂)ₘ]₂, where m=2 or 3;
   v) C[—S(CH₂)ₘ]₂, where m is as previously defined;
   vi) C═CHR₁₁, where R₁₁ is as previously defined;
   vii) C═N—O—R₁₁, where R₁₁ is as previously defined;
   viii) C═NNHR₁₁, where R₁₁ is as previously defined;
   ix) C═NNHC(O)R₁₁, where R₁₁, is as previously defined;
   x) C═NNHC(O)NHR₁₁, where R₁₁ is as previously defined;
   xi) C═NNHS(O)₂R₁₁, where R₁₁ is as previously defined;
   xii) C═NNHR₃, where R₃ is as previously defined; or
   xiii) C═NR₁₁ where R₁₁ is as previously defined;
L is
   i) —CH₃;
   ii) —CH₂CH₃;
   iii) —CH(OH)CH₃;
   iv) —C₁-C₆ alkyl, optionally substituted with one or more substituents selected from aryl, substituted aryl, heteroaryl, or substituted heteroaryl;
   v) —C₂-C₆ alkenyl, optionally substituted with one or more substituents selected from aryl, substituted aryl, heteroaryl, or substituted heteroaryl; or
   vi) —C₂-C₆ alkynyl, optionally substituted with one or more substituents selected from aryl, substituted aryl, heteroaryl, or substituted heteroaryl;
D is —N(O)CH₂—, —N(R′)C(O)—, or —N═C(OR′)—, wherein R′ is R₁₁ as previously defined;
Q is
   i) hydrogen;
   ii) —C₁-C₁₂-alkyl, C₃-C₁₂-alkenyl, or C₃-C₁₂-alkynyl, all optionally substituted with one, two or three substituents independently selected from:
      (a) halogen;
      (b) —OR₆, wherein R₆ is selected from
         1. hydrogen;
         2. —C₁-C₁₂-alkyl containing 0, 1, 2, or 3 heteroatoms selected from O, S or N, optionally substituted with one, two, or three substituents independently selected from aryl, substituted aryl, heteroaryl, or substituted heteroaryl;
         3. aryl;
         4. substituted aryl;
         5. heteroaryl; and
         6. substituted heteroaryl;
      (c) —NR₄R₅, where R₄ and R₅ are each independently R₆, where R₆ is as previously defined, or in the alternative R₄ and R₅, together with the atom to which they are attached, form a heterocycloalkyl or substituted heterocycloalkyl moiety;
      (d) ═N—O—R₆, where R₆ is as previously defined;
      (e) —R₁, where R₁ is as previously defined;
      (f) —C₃-C₈-cycloalkyl;
      (g) substituted —C₃-C₈-cycloalkyl;
      (h) heterocycloalkyl;
      (i) substituted heterocycloalkyl;
      (j) —NHC(O)R₆, where R₆ is as previously defined;
      (k) —NHC(O)OR₇, where R₇ is selected from:
         1. —C₁-C₁₂-alkyl containing 0, 1, 2, or 3 heteroatoms selected from O, S or N, optionally substituted with one, two, or three substituents independently selected from aryl, substituted aryl, heteroaryl, or substituted heteroaryl;
         2. aryl;
         3. substituted aryl;
         4. heteroaryl; or
         5. substituted heteroaryl;
      (l) —NHC(O)NR₄R₅, where R₄ and R₅ are as previously defined;
      (m) —OC(O)NR₄R₅, where R₄ and R₅ are as previously defined;
      (n) —OC(O)R₇, where R₇ is as previously defined;
      (o) —OC(O)OR₇, where R₇ is as previously defined;
      (p) —OC(O)NR₄R₅, where R₄ and R₅ are as previously defined,
      (q) —C(O)R₆, where R₆ is as previously defined,
      (r) —CO₂R₆, where R₆ is as previously defined, or
      (s) —C(O)NR₄R₅, where R₄ and R₅ are as previously defined;
X is hydrogen;
Y is
   i) hydrogen;
   ii) —OH;
   iii) —ORₚ, where Rₚ is as previously defined;
   iv) —OR₁₁, where R₁₁ is as previously defined;
   v) —OC(O)R₁₁, where R₁₁ is as previously defined;
   vi) —OC(O)NHR₁₁, where R₁₁ is as previously defined;
   vii) —S(O)ₙR₁₁, where n and R₁₁ are as previously defined; 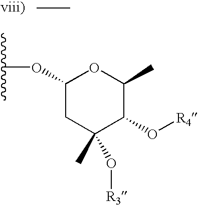
      (l) where R₃″ is hydrogen or methyl; R₄″ is hydrogen or Rₚ, where Rₚ is as previously defined; or
in the alternative, X and Y combined together are oxo;
Z is
   i) hydrogen;
   ii) methyl; or
   iii) halogen; and
R₂′ is hydrogen or Rₚ, where Rₚ, is as previously defined.
   A detailed description of the compounds of Formula 16 and their synthesis is found in U.S. Pat. No. 6,645,941, incorporated herein by reference.

In another embodiment, the present invention provides methods of treating cystic fibrosis using the compounds of Formula 17, as shown below, or a pharmaceutically acceptable salt, ester or prodrug thereof. 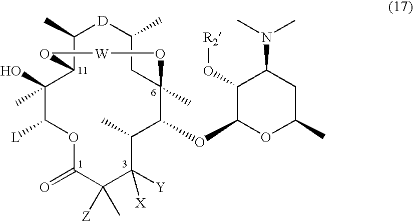
In Formula 17:
W is
   (a) —CH₂—C(A)═C(B)—CH₂—, wherein, A and B are independently selected from:
      1. hydrogen;
      2. deuterium;
      3. halogen;
      4. R₁, wherein R₁ is selected from:
         a. —C₁-C₆ alkyl, optionally substituted with one or more substituents selected from halogen, aryl, substituted aryl, heteroaryl, or substituted heteroaryl;
         b. —C₂-C₆ alkenyl, optionally substituted with one or more substituents selected from halogen, aryl, substituted aryl, heteroaryl, or substituted heteroaryl; or
         c. —C₂-C₆ alkynyl, optionally substituted with one or more substituents selected from halogen, aryl, substituted aryl, heteroaryl, or substituted heteroaryl;
      5. R₂, wherein R₂ is selected from:
         a. aryl;
         b. heteroaryl;
         c. substituted aryl;
         d. substituted heteroaryl;
         e. heterocycloalkyl; or
         f. substituted heterocycloalkyl;
      6. —(C₁-C₃-alkyl)-M-(C₁-C₃-alkyl)-R₂, wherein M=—O—, —NH—, —N(CH₃)—, —NHC(O)— or —S(O)ₙ—, wherein n=0, 1 or 2, and R₂ is as previously defined;
      7. —(C₁-C₃-alkyl)-M-R₂, wherein M and R₂ are as previously defined;
      8. —C(O)—V—R₃, wherein V is absent, O or S, and R₃ is H, R₁ or R₂; where R₁ and R₂ are as previously defined; or
      9. —C(O)—NR₁₁R₁₂, wherein R₁₁ and R₁₂ are each independently selected from:
         a. hydrogen;
         b. —C₁-C₆-alkyl, optionally substituted with one or more substituents selected from halogen, aryl, substituted aryl, heteroaryl, or substituted heteroaryl;
         c. —C₂-C₆-alkenyl, optionally substituted with one or more substituents selected from halogen, aryl, substituted aryl, heteroaryl, or substituted heteroaryl;
         d. —C₂-C₆-alkynyl, optionally substituted with one or more substituents selected from halogen, aryl, substituted aryl, heteroaryl, or substituted heteroaryl; or
            in the alternative, R₁ and R₁₂ taken together with the nitrogen atom to which they are connected form a 3- to 7-membered ring which may optionally contain one or more double bonds and one or more heterofunctions selected from —O—, —NH—, —N(C₁-C₆-alkyl)-, —N(R₂)—, —S(O)ₙ—, wherein n and R₂ are as previously defined;
   (b) —CH₂—CH(A)-C(B)═CH—, wherein A and B are as previously defined;
   (c) —CH₂—CH(E)-CH(G)-CH₂—, wherein E and G are independently selected from
      1. A, wherein A is as previously defined;
      2. —OH;
      3. —OR^{p}, wherein R^{p} is a hydroxy protecting group;
      4. —O—R₉, wherein R₉ is R₁ or R₂, and wherein R₁ and R₂ are as previously defined;
      5. —S(O)ₙR₉, wherein n and R₉ are as previously defined;
      6. —NHC(O)R₃, wherein R₃ is as previously defined;
      7. —NHC(O)NR₁₁R₃, wherein R₁₁ and R₃ are as previously defined;
      8. —NHS(O)₂R₉, wherein R₉ is as previously defined;
      9. —NHR₁₃, wherein R₁₃ is an amino protecting group; or
      10. —NR₁₁R₁₂, wherein R₁₁ and R₁₂ are as previously defined; 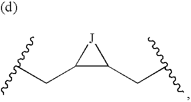
      wherein -J- is selected from —O—; —O—C(O)—CH(R₇)—; —N(R₇)—; —O—C(O)—N(R₇)—; —O—C(O)—O—; —N(R₇)—N═N—; —C(R₇)═N—O—; or —CH(R₇)—N(R₈)—O—; wherein R₇ and R₈ are independently selected from R₃, wherein R₃ is as previously defined; or, in the alternative, -J- is taken with the two carbon atoms to which it is attached to form a cyclic moiety selected from
         a. C₃-C₁₂ cycloalkyl;
         b. C₃-C₁₂ cycloalkenyl; or
         c. heterocycloalkyl; or
   (e) —CH₂—C(R₄)(R₅)—CH₂—CH₂—, wherein R₄ and R₅ taken together with the carbon atom to which they are attached are selected from:
      1. C═O;
      2. C(OR₁)₂, wherein R₁ is as previously defined;
      3. C(SR₁)₂, wherein R₁ is as previously defined;
      4. C(OR₁₂)(OR₁₃), where R₁₂ and R₁₃ taken together are —(CH₂)ₘ—, and where m is 2 or 3;
      5. C(SR₁₂)(SR₁₃), where R₁₂ and R₁₃ taken together are —(CH₂)ₘ—, where m is as previously defined;
      6. C═CHR₃, wherein R₃ is as previously defined;
      7. C═N—O—R₃, wherein R₃ is as previously defined;
      8. C═NNHR₃, wherein R₃ is as previously defined;
      9. C═NNHC(O)R₃, wherein R₃ is as previously defined;
      10. C═NNHC(O)NR₁₁R₃, wherein R₁₁ and R₃ are as previously defined;
      11. C═NNHS(O)₂R₉, wherein R₉ is as previously defined;
      12. C═NNHR₁₃, wherein R₁₃ is as previously defined; or
      13. C═NR₉, wherein R₉ is as previously defined;
L is
   (a) —CH₃;
   (b) —CH₂CH₃;
   (c) —CH(OH)CH₃;
   (d) —C₁-C₆ alkyl, optionally substituted with one or more substituents selected from aryl, substituted aryl, heteroaryl, or substituted heteroaryl;
   (e) —C₂-C₆ alkenyl, optionally substituted with one or more substituents selected from aryl, substituted aryl, heteroaryl, or substituted heteroaryl; or
   (f) —C₂-C₆ alkynyl, optionally substituted with one or more substituents selected from aryl, substituted aryl, heteroaryl, or substituted heteroaryl; or
D is —N(O)CH₂—, —N(R′)C(O)—, or —N═C(OR′)—, wherein R′ is R₁₁ as previously defined;
Q is
   (a) hydrogen;
   (b) —C₁-C₁₂-alkyl, C₂-C₁₂-alkenyl, or C₂-C₁₂-alkynyl, all optionally substituted with one, two or three substituents independently selected from:
      1. halogen;
      2. —OR₆, wherein R₆ is selected from
         a. hydrogen;
         b. —C₁-C₁₂-alkyl containing 0, 1, 2, or 3 heteroatoms selected from O, S or N, optionally substituted with one, two, or three substituents independently selected from aryl, substituted aryl, heteroaryl, or substituted heteroaryl;
         c. aryl;
         d. substituted aryl;
         e. heteroaryl;
         f. substituted heteroaryl;
         g. heterocycloalkyl; or
         h. substituted heterocycloalkyl;
      3. —NR₄R₅, where R₄ and R₅ are each independently R₆, where R₆ is as previously defined, or in the alternative R₄ and R₅, together with the atom to which they are attached, form a heterocycloalkyl or substituted heterocycloalkyl moiety;
      4. ═N—O—R₆, where R₁ is as previously defined;
      5. —R₁, where R₁ is as previously defined;
      6. —C₃-C₁₂-cycloalkyl;
      7. substituted —C₃-C₁₂-cycloalkyl;
      8. heterocycloalkyl;
      9. substituted heterocycloalkyl;
      10. —NHC(O)R₆, where R₆ is as previously defined;
      11. —NHC(O)OR₇, where R₇ is selected from:
         a. —C₁-C₁₂-alkyl containing 0, 1, 2, or 3 heteroatoms selected from O, S or N, optionally substituted with one, two, or three substituents independently selected from aryl, substituted aryl, heteroaryl, or substituted heteroaryl;
         b. aryl;
         c. substituted aryl;
         d. heteroaryl;
         e. substituted heteroaryl;
         f. heterocycloalkyl; or
         g. substituted heterocycloalkyl;
      12. —NHC(O)NR₄R₅, where R₄ and R₅ are as previously defined;
      13. —OC(O)NR₄R₅, where R₄ and R₅ are as previously defined;
      14. —OC(O)R₇, where R₇ is as previously defined;
      15. —OC(O)OR₇, where R₇ is as previously defined;
      16. —OC(O)NR₄R₅, where R₄ and R₅ are as previously defined,
      17. —C(O)R₆, where R₆ is as previously defined,
      18. —CO₂R₆, where R₆ is as previously defined, or
      19. —C(O)NR4R₅, where R₄ and R₅ are as previously defined;
X is hydrogen;
Y is
   (a) hydrogen;
   (b) —OH;
   (c) —ORₚ, where Rₚ is as previously defined;
   (d) —OR_{y}, where R_{y} is R₁ and R₂ as previously defined;
   (e) —OC(O)R_{y}, where R_{y} is R₁ and R₂ as previously defined;
   (f) —OC(O)NHR_{y}, where R_{y} is R₁ and R₂ as previously defined;
   (g) —S(O)ₙR_{y}, where n is previously defined and Ry is R₁ and R₂ as previously defined; 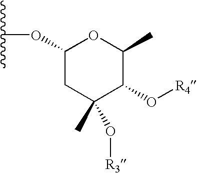
   where R₃″ is hydrogen or methyl; R₄″ is hydrogen or Rₚ, where Rₚ is as previously defined; or
   (h) in the alternative, X and Y combined together are oxo;
Z is
   (a) hydrogen;
   (b) methyl; or
   (c) halogen; and
R₂′ is hydrogen or Rₚ, where Rₚ, is as previously defined.
   A detailed description of the compounds of Formula 17 and their synthesis is found in U.S. Pat. No. 6,764,998, U.S. Ser. No. 10/758,409, filed Jan. 14, 2004, and U.S. Ser. No. 10/763,377, filed Jan. 23, 2004, all incorporated herein by reference.

In another embodiment, the present invention provides methods of treating cystic fibrosis using the compounds of Formulas 18-21 as shown below, or a pharmaceutically acceptable salt, ester or prodrug thereof.

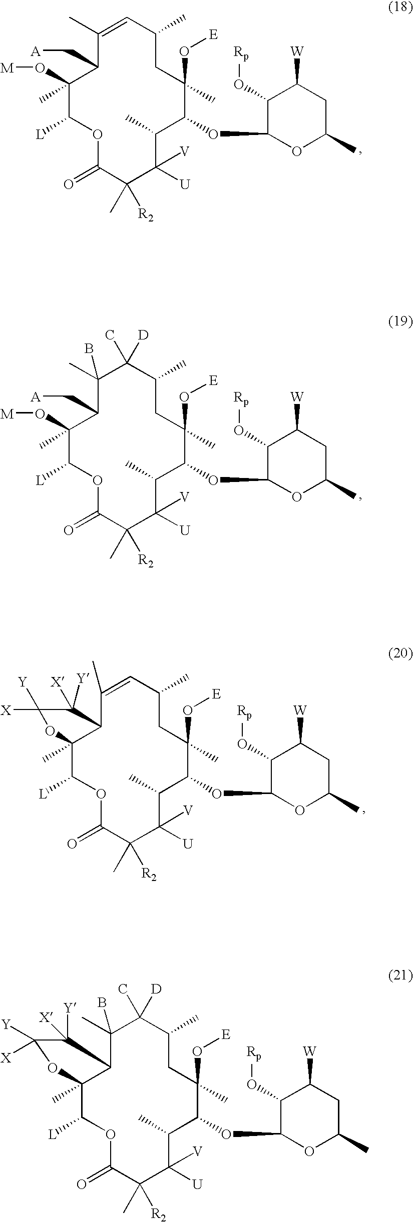

In Formulas 18-21:
A is independently selected from R₁, —CH(R_{A1})(R_{A2});
Each B, C, and D is independently selected from deuterium, halogen, R₁, OR₁, S(O)ₙR₁, —NR₁C(O)R₁, —NR₁C(O)NR₃R₄, —NR₁S(O)ₙR₁, —C(O)NR₃R₄, and —NR₃R₄;
or B and C taken together are selected from —O—, —S—, —N(R₁)—, a substituted or unsubstituted alicyclic group, or a substituted or unsubstituted heterocyclic group;
or C and D taken together with the carbon atom to which they are attached are selected CO, C═CHR₁, C═NR₁, C═NOR₁, C═NO(CH₂)ₘR₁, C═NNHR₁, C═NNHCOR₁, C═NNHCONR₃R₄, C═NNHS(O)ₙR₁, C═N—N═CHR₁;
E is independently selected from R₁;
Each R₁ is independently selected from the group consisting of: hydrogen, acyl, silane, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group;
Each of R₃ and R₄ is independently selected from the group consisting of: hydrogen, acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, a substituted, or unsubstituted heterocyclic group; or can be taken together with the nitrogen atom to which they are attached to form a substituted or unsubstituted heterocyclic or heteroaromatic ring;
Each R_{A1} and R_{A2} are independently selected from hydrogen, deuterium, halogen, R₁, OR₁, S(O)ₙR, —NR₁C(O)R₁, —NR₁C(O)NR₃R₄, —NHS(O)ₙR₁, —CONR₃R₄, and NR₃R₄;
or R_{A1} and R_{A2}, taken together with the carbon atom to which they are attached, form a substituted or unsubstituted alicyclic, aromatic, heterocyclic or heteroaromatic ring;
or R_{A1} and R_{A2} taken together with the carbon atom to which they are attached are selected from C═O, C═CHR₁, C═NR₁, C═NOR₁, C═NO(CH₂)ₘR₁, C═NNHR₁, C═NNHCOR₁, C═NNHCONR₃R₄, C═NNHS(O)ₙR₁, C═N—N═CHR₁;
M is selected from the group consisting of: R₁, C(O)R₁, S(O)ₙR₁, or C(O)NR₃R₄;
L is selected from the group consisting of: hydrogen, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group;
X, Y, X′, and Y′ are independently selected from the group consisting of: deuterium, halogen, R₁, OR₁, S(O)ₙR₁, —NR₁C(O)R₁, —NR₁C(O)NR₃R₄, —NR₁S(O)ₙR₁, —C(O)NR₃R₄, and —NR₃R₄;
or X and Y, taken together with the carbon atom to which they are attached, are C═O;
or X and X′, taken together with the carbon atoms to which they are attached, are C═C;
one of U or V is hydrogen and the other is independently selected from the group consisting of: R₁, OR₁, OC(O)R₁, OC(O)NR₃R₄, S(O)ₙR₁, or 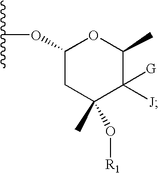
or U and V, taken together with the carbon atom to which they are attached, are C═O;
one of J or G is hydrogen and the other is selected from: R₁, OR₁, or NR₃R₄;
or, J and G, taken together with the carbon atom to which they are attached, are selected from: C═O, C═NR₁, C═NOR₁, C═NO(CH₂)ₘR₁, C═NNHR₁, C═NNHCOR₁, C═NNHCONR₁R₂, C═NNHS(O)ₙR₁, or C═N—N═CHR₁;
W is NR₃R₄;
R₂ is selected from hydrogen, alkyl or halogen;
Rₚ is selected from a hydrogen and a hydroxy protecting group;
m is an integer; and
n is 0, 1, or 2.
   A detailed description of the compounds of Formulas 18-21, and their synthesis is found in U.S. Ser. No. 10/745,856, incorporated herein by reference.

### DETAILED DESCRIPTION OF THE INVENTION

Definitions

Listed below are definitions of various terms used to describe this invention. These definitions apply to the terms as they are used throughout this specification and claims, unless otherwise limited in specific instances, either individually or as part of a larger group.

An “aliphatic group” is non-aromatic moiety that may contain any combination of carbon atoms, hydrogen atoms, halogen atoms, oxygen, nitrogen, sulfur or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

Suitable aliphatic or aromatic substituents include, but are not limited to, —F, —Cl, —Br, —I, —OH, protected hydroxy, aliphatic ethers, aromatic ethers, oxo, —NO₂, —CN, —C₁-C₁₂-alkyl optionally substituted with halogen (such as perhaloalkyls), C₂-C₁₂-alkenyl optionally substituted with halogen, —C₂-C₁₂-alkynyl optionally substituted with halogen, —NH₂, protected amino, —NH —C₁-C₁₂-alkyl, —NH —C₂-C₁₂-alkenyl, —NH —C₂-C₁₂-alkenyl, —NH —C₃-C₁₂-cycloalkyl, —NH-aryl, —NH-heteroaryl, —NH —heterocycloalkyl, -dialkylamino, -diarylamino, -diheteroarylamino, —O—C₁-C₁₂-alkyl, —O—C₂-C₁₂-alkenyl, —O—C₂-C₁₂-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkynyl, —C(O)—C₃-C₁₂-cycloalkyl, —C(O)-aryl, —C(O)-heteroaryl, —C(O)—heterocycloalkyl, —CONH₂, —CONH—C₁-C₁₂-alkyl, —CONH—C₂-C₁₂-alkenyl, —CONH—C₂-C₁₂-alkynyl, —CONH—C₃-C₁₂-cycloalkyl, —CONH-aryl, —CONH-heteroaryl, —CONH-heterocycloalkyl, —CO₂—C₁-C₁₂-alkyl, —CO₂—C₂-C₁₂-alkenyl, —CO₂—C₂-C₁₂-alkynyl, —CO₂—C₃-C₁₂-cycloalkyl, —CO₂-aryl, —CO₂-heteroaryl, —CO₂-heterocycloalkyl, —OCO₂—C₁-C₁₂-alkyl, —OCO₂—C₂-C₁₂-alkenyl, —OCO₂—C₂-C₁₂-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkynyl, —OCONH—C₃-C₁₂-cycloalkyl, —OCONH— aryl, —OCONH—heteroaryl, —OCONH— heterocycloalkyl, —NHC(O)—C₁-C₁₂-alkyl, —NHC(O)—C₂-C₁₂-alkenyl, —NHC(O)—C₂-C₁₂-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkynyl, —NHCO₂—C₃-C₁₂-cycloalkyl, —NHCO₂— aryl, —NHCO₂— heteroaryl, —NHCO₂— heterocycloalkyl, —NHC(O)NH₂, NHC(O)NH—C₁-C₁₂-alkyl, —NHC(O)NH—C₂-C₂-alkenyl, —NHC(O)NH—C₂-C₁₂-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, NHC(S)NH—C₁-C₁₂-alkyl, —NHC(S)NH—C₂-C₁₂-alkenyl, —NHC(S)NH—C₂-C₁₂-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, NHC(NH)NH—C₁-C₂-alkyl, —NHC(NH)NH—C₂-C₁₂-alkenyl, —NHC(NH)NH—C₂-C₁₂-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, NHC(NH)—C—C₁₂-alkyl, —NHC(NH)—C₂-C₁₂-alkenyl, —NHC(NH)—C₂-C₁₂-alkynyl, —NHC(NH)—C₃-C₂-cycloalkyl, —NHC(NH)-aryl, —NHC(NH)-heteroaryl, —NHC(NH)-heterocycloalkyl, —C(NH)NH—C₁-C₁₂-alkyl, —C(H)NH—C₂-C₁₂-alkenyl, —C(NMNH—C₂-C₂-alkynyl, —C(NH)NH—C₃-C₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkynyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkynyl, —SO₂NH—C₃-C₁₂-cycloalkyl, —SO₂NH— aryl, —SO₂NH— heteroaryl, —SO₂NH— heterocycloalkyl, —NHSO₂—C₁-C₁₂-alkyl, —NHSO₂—C₂-C₂-alkenyl, —NHSO₂—C₂-C₁₂-alkynyl, —NHSO₂-C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkynyl, —S—C₃-C₁₂-cycloalkyl, —S-aryl, —S-heteroaryl, —S-heterocycloalkyl, or methylthiomethyl. It is understood that the aryls, heteroaryls, alkyls and the like can be further substituted.

The terms “C₁-C₃ alkyl,” “C₁-C₆ alkyl,” or “C₁-C₁₂ alkyl,” as used herein, refer to saturated, straight- or branched-chain hydrocarbon radicals containing between one and three, one and twelve, or one and six carbon atoms, respectively. Examples of C₁-C₃ alkyl radicals include methyl, ethyl, propyl and isopropyl radicals; examples of C₁-C₆ alkyl radicals include, but are not limited to, methyl, ethyl, propyl, isopropyl, n-butyl, tert-butyl, neopentyl and n-hexyl radicals; and examples of C₁-C₁₂ alkyl radicals include, but are not limited to, ethyl, propyl, isopropyl, n-hexyl, octyl, decyl, dodecyl radicals.

The term “substituted alkyl,” as used herein, refers to a “C₂-C₁₂ alkyl” or “C₁-C₆ alkyl” group as previously defined, substituted by one, two, three or more aliphatic substituents.

The terms “C₂-C₁₂ alkenyl” or “C₂-C₆ alkenyl,” as used herein, denote a monovalent group derived from a hydrocarbon moiety containing from two to twelve or two to six carbon atoms having at least one carbon-carbon double bond by the removal of a single hydrogen atom. Alkenyl groups include, but are not limited to, for example, ethenyl, propenyl, butenyl, 1-methyl-2-buten-1-yl, alkadienes and the like.

The term “substituted alkenyl,” as used herein, refers to a “C₂-C₁₂ alkenyl” or “C₂-C₆ alkenyl” group as previously defined, substituted by one, two, three or more aliphatic substituents.

The terms “C₂-C₁₂ alkynyl” or “C₂-C₆ alkynyl,” as used herein, denote a monovalent group derived from a hydrocarbon moiety containing from two to twelve or two to six carbon atoms having at least one carbon-carbon triple bond by the removal of a single hydrogen atom. Representative alkynyl groups include, but are not limited to, for example, ethynyl, 1-propynyl, 1-butynyl, and the like.

The term “substituted alkynyl,” as used herein, refers to a “C₂-C₁₂ alkynyl” or “C₂-C₆ alkynyl” group as previously defined, substituted by one, two, three or more aliphatic substituents.

The term “C₁-C₆ alkoxy,” as used herein, refers to a C₁-C₆ alkyl group, as previously defined, attached to the parent molecular moiety through an oxygen atom. Examples of C₁-C₆-alkoxy include, but are not limited to, methoxy, ethoxy, propoxy, isopropoxy, n-butoxy, sec-butoxy, tert-butoxy, n-pentoxy, neopentoxy and n-hexoxy.

The terms “halo” and “halogen,” as used herein, refer to an atom selected from fluorine, chlorine, bromine and iodine.

The terms “aryl” or “aromatic” as used herein, refer to a mono- or bicyclic carbocyclic ring system having one or two aromatic rings including, but not limited to, phenyl, naphthyl, tetrahydronaphthyl, indanyl, idenyl and the like.

The terms “substituted aryl” or “substituted aromatic,” as used herein, refer to an aryl or aromatic group substituted by one, two, three or more aromatic substituents.

The term “arylalkyl,” as used herein, refers to an aryl group attached to the parent compound via a C₁-C₃ alkyl or C₁-C₆ alkyl residue. Examples include, but are not limited to, benzyl, phenethyl and the like.

The term “substituted arylalkyl,” as used herein, refers to an arylalkyl group, as previously defined, substituted by one, two, three or more aromatic substituents.

The terms “heteroaryl” or “heteroaromatic,” as used herein, refer to a mono-, bi-, or tri-cyclic aromatic radical or ring having from five to ten ring atoms of which at least one ring atom is selected from S, O and N; zero, one or two ring atoms are additional heteroatoms independently selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. The heteroaromatic ring may be bonded to the chemical structure through a carbon or hetero atom.

The terms “substituted heteroaryl” or “substituted heteroaromatic,” as used herein, refer to a heteroaryl or heteroaromatic group, substituted by one, two, three, or more aromatic substituents.

The term “alicyclic,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo [2.2.1]heptyl, and bicyclo [2.2.2] octyl.

The term “substituted alicyclic,” as used herein, refers to an alicyclic group substituted by one, two, three or more aliphatic substituents.

The term “heterocyclic,” as used herein, refers to a non-aromatic ring, comprising three or more ring atoms, or a bi- or tri-cyclic group fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, (iv) any of the above rings may be fused to a benzene ring, and (v) the remaining ring atoms are carbon atoms which may be optionally oxo-substituted. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl.

The term “substituted heterocyclic,” as used herein, refers to a heterocyclic group, as previously defined, substituted by one, two, three or more aliphatic substituents.

The term “heteroarylalkyl,” as used herein, to an heteroaryl group attached to the parent compound via a C₁-C₃ alkyl or C₁-C₆ alkyl residue. Examples include, but are not limited to, pyridinylmethyl, pyrimidinylethyl and the like.

The term “substituted heteroarylalkyl,” as used herein, refers to a heteroarylalkyl group, as previously defined, substituted by independent replacement of one, two, or three or more aromatic substituents.

The term “alkylamino” refers to a group having the structure —NH(C₁-C₁₂ alkyl).

The term “dialkylamino” refers to a group having the structure —N(C₁-C₁₂ alkyl) (C₁-C₁₂ alkyl) and cyclic amines. Examples of dialkylamino are, but not limited to, dimethylamino, diethylamino, methylethylamino, piperidino, morpholino and the like.

The term “alkoxycarbonyl” represents an ester group, i.e., an alkoxy group, attached to the parent molecular moiety through a carbonyl group such as methoxycarbonyl, ethoxycarbonyl, and the like.

The term “carboxaldehyde,” as used herein, refers to a group of formula —CHO.

The term “carboxy,” as used herein, refers to a group of formula —COOH.

The term “carboxamide,” as used herein, refers to a group of formula —C(O)NH(C₁-C₁₂ alkyl) or —C(O)N(C₁-C₁₂ alkyl) (C₁-C₁₂ alkyl), —C(O)NH₂, NHC(O)(C₁-C₁₂ alkyl), N(C₁-C₁₂ alkyl)C(O)(C₁-C₁₂ alkyl) and the like.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl (Ac or —C(O)CH₃), benzoyl (Bz or —C(O)C₆H₅), and trimethylsilyl (TMS or —Si(CH₃)₃).

The term “protected hydroxy,” as used herein, refers to a hydroxy group protected with a hydroxy protecting group, as defined above, including benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl (Ac or —C(O)CH₃), benzoyl (Bz or —C(O)C₆H₅), and trimethylsilyl (TMS or —Si(CH₃)₃).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “protected amino,” as used herein, refers to an amino group protected with an amino protecting group as defined above.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates.

The term “sugar-containing moiety” refers to a chemical moiety comprising at least one sugar group, preferably at least one deoxy sugar group, including, but not limited to for example, amino sugars such as desosamine, forosamine and derivatives thereof, and neutral sugars such as cladinose and derivatives thereof; attached directly or indirectly to the macrolide lactone ring via for example, a glycosidic bond, CH₂ or NH group, or an oxygen or sulfur atom.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein.

The compounds used in this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)— or (S)—, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters of the compounds formed by the process of the present invention which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of Formulas 1-21 which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound represented by any one of Formulas 1-21. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

This invention also encompasses pharmaceutical compositions containing pharmaceutically acceptable prodrugs of any one of compounds represented by Formulas 1-21. For example, compounds having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of Formulas 1-21. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

Unless otherwise defined, all technical and scientific terms used herein are accorded the meaning commonly known to one with ordinary skill in the art.

Pharmaceutical Compositions.

The pharmaceutical compositions of the present invention comprise a therapeutically effective amount of at least one compound of Formulas 1-21 formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminun hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals orally, rectally, parenterally, intracisternally, intravaginally, intraperitoneally, topically (as by powders, ointments, or drops), buccally, as an oral or nasal spray, or via pulmonary delivery.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethyl cellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The total daily dose of the compounds of this invention administered to a human or other animal via one of the delivery routes discussed above, in single or in divided doses can be in amounts, for example, from 0.01 to about 50 mg/kg body weight or more usually from 0.1 to about 25 mg/kg body weight. Single dose compositions may contain such amounts or submultiples thereof to make up the daily dose. In general, treatment regimens according to the present invention comprise administration to a patient in need of such treatment of from about 10 mg to about 1000 mg of the compound(s) of this invention per day in single or multiple doses.

In one preferred embodiment therapeutic compositions of the invention are administered by pulmonary delivery. For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. In general, particles ranging from about 1 to 10 microns in size are within the respirable range. The therapeutic compositions containing at least one compound of Formulas 1-21, are preferably administered by direct inhalation into the respiratory system for delivery as a mist or other aerosol or dry powder. Particles of non-respirable size which are included in the aerosol tend to be deposited in the throat and swallowed; thus the quantity of non-respirable particles in the aerosol is preferably minimized. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43,650 by Montgomery, all of which are incorporated herein by reference). A discussion of pulmonary delivery of antibiotics is also found in U.S. Pat. No. 6,014,969, incorporated herein by reference.

The dosage of active compound via the pulmonary route of delivery will vary depending on the condition being treated and the state of the subject, but generally may be an amount sufficient to achieve dissolved concentrations of active compound on the airway surfaces of the subject. Depending upon the solubility of the particular formulation of active compound administered, the daily dose may be divided among one or several unit dose administrations. The daily dose by weight will depend upon the age and condition of the subject. Such a daily dose of active compound ranges from about 0.20 mg/kg per day to about as 2.0 mg per day, and more preferably from about 0.1 to about 1 mg/kg and most preferably from about 0.200 mg/kg to about 0.650 mg/kg. The doses of the active compound may be provided as one or several prepackaged units.

Therapeutic Uses

In accordance with the invention, methods are provided for treating CF in a patient in need thereof comprising administering to the patient a therapeutically effective amount of at least one compound of Formulas 1-21. As used herein the terms “treating” or “treatment” include i) prophylactic treatment of those patients susceptible to the symptoms CF including pulmonary infection and inflammation associated with CF, ii) treatment at the initial onset of symptoms of pulmonary infection and inflammation associated with CF, and iii) treatment of ongoing or relapsing symptoms of infection and inflammation associated with CF. A therapeutically effective amount is an amount sufficient to prevent, diminish or eradicate symptoms of CF including chronic pulmonary inflammation and infection.

In one embodiment the invention provides a co-therapeutic treatment regimen for treating CF. As used herein a “co-therapeutic treatment regimen” means a treatment regimen wherein two or more drugs are administered simultaneously or sequentially, separated by minutes, hours or days, but in some way act together to provide the desired therapeutic response. In one embodiment, a co-therapeutic treatment regimen for treating CF in a patient comprises, administering to the patient, a therapeutically effective amount at least one compound of Formulas 1-21 in combination with a therapeutically effective amount of at least one other therapeutic agent that is active against CF and its associated symptoms. Examples of therapeutic agents that are active against CF include but are not limited to i) macrolide antibiotics other than those described herein such as erythromycin, leucomycin, azithromycin and clarithromycin, ii) ceftazidime, iii) quinolones, iv) other agents that interfere with protein synthesis such as chloramphenicol, and tetracycline, or v) aminoglycosides such as gentamicin and tobramycin.

The invention further provides methods of treating patients suffering from an inflammatory condition comprising administering to a patient in need thereof, a therapeutically effective amount of at least one compound of Formulas 1-21. Inflammatory conditions usually are characterized by an activation of the immune system, such as T-cell activation. Such activation often is mediated by effector cells, such as neutrophils, macrophages or eosinophils, and may be promoted by specific mediators such as IL-1, Il-4 or TNF or allergic antibodies. Some inflammatory conditions involve cell-mediated granulomatous inflammation. Thus, a patient in this context often will suffer from a disorder characterized by one or more of the foregoing signs of an inflammatory condition. Specific examples of inflammatory conditions treatable according to the invention include, but are not limited to, scleritis; epi-scleritis; allergic conjunctivitis; pulmonary inflammatory diseases, particularly CF, asthma, chronic obstructive pulmonary disease (COPD), allergic bronchopulmonary aspergillosis (ABPA), and sarcoidosis; procto-sigmoiditis; allergic rhinitis; arthritis; tendonitis; apthous stomatitis; and inflammatory bowel disease.

Other diseases related to inflammation include but are not limited to, inflammatory and hyperproliferative skin diseases and cutaneous manifestations of immunologically-mediated illnesses, such as psoriasis, atopical dermatitis, contact dermatitis and further eczematous dermatitises, seborrhoeis dermatitis, Lichen planus, Pemphigus, bullous pemphigoid, Epidermolysis bullosa, urticaria, angioedemas, vasculitides, erythemas, cutaneous eosinophilias, Lupus erythematosus, acne and Alopecia greata; various eye diseases (autoimmune and otherwise) such as keratoconjunctivitis, vernal conjunctivitis, keratitis, herpetic keratitis, conical cornea, dystrophia epithelialis corneae, corneal leukoma, ocular pemphigus, Mooren's ulcer, Scleritis, Graves' opthalmopathy, Vogt-Koyanagi-Harada syndrome, sarcoidosis, multiple myeloma, etc.; obstructive airway diseases, which includes conditions such as chronic obstructive pulmonary disease (COPD), asthma (for example, bronchial asthma, allergic asthma, intrinsic asthma, extrinsic asthma and dust asthma), particularly chronic or inveterate asthma (for example, late asthma and airway hyper-responsiveness), bronchitis, allergic rhinitis and the like; inflammation of mucosa and blood vessels such as gastric ulcers, vascular damage caused by ischemic diseases and thrombosis.

Other treatable inflammatory conditions include but are not limited to ischemic bowel diseases, inflammatory bowel diseases, necrotizing enterocolitis, intestinal lesions associated with thermal burns and leukotriene β₄-mediated diseases; intestinal inflammations/allergies such as Coeliac diseases, proctitis, eosinophilic gastroenteritis, mastocytosis, Crohn's disease and ulcerative colitis; food-related allergic diseases which have symptomatic manifestation remote from the gastro-intestinal tract (e.g., migraine, rhinitis and eczema).

The invention further provides a co-therapeutic treatment regimen for treating an inflammatory condition. In one embodiment, a co-therapeutic treatment regimen for treating inflammation in patient comprises administering to the patient, a therapeutically effective amount at least one compound of Formulas 1-21 in combination with a therapeutically effective amount of at least one other therapeutic anti-inflammatory agent. Examples of anti-inflammatory agents include but are not limited to non-steroidal anti-inflammatory agents such as indomethacin, ketoprofen, celcoxib, rofecoxib, meclofenamic acid, fenoprofen, diflunisal, tolfenamic acid, naproxen, ibuprofen, flurbiprofen, or nabumetone.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for treating cystic fibrosis in a patient in need thereof comprising administering to the patient a therapeutically effective amount of at least one compound of Formulas 1-21.

2. A co-therapeutic treatment regimen for treating cystic fibrosis in a patient comprising administering to the patient, a therapeutically effective amount at least one compound of Formulas 1-21 in combination with a therapeutically effective amount of at least one other therapeutic agent that is active against cystic fibrosis.

3. The method of claim 2 wherein the therapeutic agent that is active against cystic fibrosis is an agent that interferes with protein synthesis in a pathogen or an aminoglycoside.

4. The method of claim 3 wherein the therapeutic agent that is active against cystic fibrosis is erythromycin, azithromycin, clarithromycin, leucomycin, chloramphenicol, tetracycline, or tobramycin.

5. The method of claim 2 wherein the therapeutic agent that is active against cystic fibrosis is ceftazidime, or quinolones.

6. A method of treating patients suffering from an inflammatory condition comprising administering to a patient in need thereof, a therapeutically effective amount of at least one compound of Formulas 1-21.

7. The method of claim 6 wherein the inflammatory condition is selected from the group consisting of scleritis; epi-scleritis; allergic conjunctivitis; pulmonary inflammatory diseases, particularly CF, asthma, chronic obstructive pulmonary disease (COPD), allergic bronchopulmonary aspergillosis (ABPA), and sarcoidosis; procto-sigmoiditis; allergic rhinitis; arthritis; tendonitis; apthous stomatitis; and inflammatory bowel disease.

8. A co-therapeutic treatment regimen for treating an inflammatory condition in a patient comprising administering to the patient, a therapeutically effective amount at least one compound of Formulas 1-21 in combination with a therapeutically effective amount of at least one other therapeutic agent that is active against inflammation.

9. The method of claim 8 wherein the therapeutic agent that is active against inflammation is a non-steroidal anti-inflammatory agent.

10. The method of claim 9 wherein the non-steroidal anti-inflammatory agent is indomethacin, ketoprofen, celcoxib, rofecoxib, meclofenamic acid, fenoprofen, diflunisal, tolfenamic acid, naproxen, ibuprofen, flurbiprofen, or nabumetone.
